SEQUENCE LISTING (29 sequences)

SEQ ID NO: 1 - protein, 636 aa, Unknown
  Carbon monoxide dehydrogenase II derived from Carboxydothermus hydrogenoformans
MAKQNLKSTDRAVQQMLDKAKREGIQTVWDRYEAMKPQCGFGETGLCCRHCLQGPCRINPFGDEPKVGICGATAEVIVARGLDRSIAAGAAGHSGHAKHLAHTLKKAVQGKAASYMIKDRTKLHSIAKRLGIPTEGQKDEDIALEVAKAALADFHEKDTPVLWVTTVLPPSRVKVLSAHGLIPAGIDHEIAEIMHRTSMGCDADAQNLLLGGLRCSLADLAGCYMGTDLADILFGTPAPVVTESNLGVLKADAVNVAVHGHNPVLSDIIVSVSKEMENEARAAGATGINVVGICCTGNEVLMRHGIPACTHSVSQEMAMITGALDAMILDYQCIQPSVATIAECTGTTVITTMEMSKITGATHVNFAEEAAVENAKQILRLAIDTFKRRKGKPVEIPNIKTKVVAGFSTEAIINALSKLNANDPLKPLIDNVVNGNIRGVCLFAGCNNVKVPQDQNFTTIARKLLKQNVLVVATGCGAGALMRHGFMDPANVDELCGDGLKAVLTAIGEANGLGGPLPPVLHMGSCVDNSRAVALVAALANRLGVDLDRLPVVASAAEAMHEKAVAIGTWAVTIGLPTHIGVLPPITGSLPVTQILTSSVKDITGGYFIVELDPETAADKLLAAINERRAGLGLPW

SEQ ID NO: 2 - protein, 633 aa, Unknown
  Carbon monoxide dehydrogenase IV derived from Carboxydothermus hydrogenoformans
MDKSKLSVDPVIPNLYRKAREEGISTVFDRYEAQQPQCGFGLTGLCCRHCVQGPCRIDPFGEGPQAGICGATAEVITARNLLRQVTAGAAAHVDHAYDVLEVLEQIAQGTESYSIKDQEKLKQVAFTLGIDTANKTEQEIVEEMCQIIYRDFANSGATPMTYLKANSPRERLETWEKLGVLPRNPDREIREALHQTTMGMDADPVNLILKTIRLGLVDGFAGLKLATDLQDIIFGTPQPVVTEANLGVLKEDYVNIIVHGHVPLLSEKIVEWSRKLEDEAKKAGAKGINLAGICCTGNEVLMRQGVPLATNFLAQELAIITGAVDLMVVDVQCIMPSLAEIAACYHTRLVTTMPIVKIPGAEHVPFTTETADEASQQIVRMAIESYQKRNPAKVYIPREKAKVVAGFSVEAIVKALAKLNPDDPLKPLIDNIVSGNILGVVATVGCNNVKVKHDWFHIELVKELIKNNVLVVTTGCSAHALAKAGLMDPAAAEWAGEGLRAVLTAIGTANDLGGPLPPVLHMGSCVDNSRIGDLVIAVANYLKVSPKDLPIAASAPEYQHEKALSIGTWAVAMGIMTHLGVVPPVVGSSKVTRILTQDAEALIGGKFYVETDPYKAAAGIIEHIKAKRALLNL

SEQ ID NO: 3 - protein, 630 aa, Thermococcus sp.
MAGKKVPSKQVSITPGVGKLIEKAEEDGVKTAWHRFLEQQPQCGFGLLGVCCKNCTMGPCRIDPFGVGPTKGVCGADADTIVARNIVRMIAAGTAGHSDHSRDVVHVFKGIAEGKFKDYKLTDVEKLKELAKILGVETEGKSENEIALEVAHILEMEFGKQDEEPVRLLAATAPKKRIKVWEKLGVLPRAIDREICLSMHRTHIGCDADPASLLLHGVRTALADGWCGSMMATYLSDILFGTPKPIKSLANLGVLKEDMVNIIVHGHNPILSMKIAEIAQSEEMQKLAEQYGAKGINVAGMCCTGNEVLSRMGVQVAGNFLMQELAIITGAVEAVIVDYQCLMPSLVDVASCYHTKIITTEPKARIPGAIHVEFEPEKADEIAKEIIKIAIENYKNRVPAKVYIPEHKMELVAGFSVEAILEALGGTLEPLIKALQDGTIKGIVGIVGCNNPRVKQNYGHVTLAKELIKRDILVVGTGCWGIAAAMHGLLTPEAAEMAGPGLKAVCEALGIPPCLHMGSCVDCSRILLVLSALANALNVDISDLPVAGSAPEWMSEKAVAIGTYFVASGVFTHLGVIPPVLGSQKVTKLLTDDIEDLLGGKFYVETDPVKAAETIYNVIIEKRKKLGWPI

SEQ ID NO: 4 - protein, 989 aa, Methylobacterium extorquens
MSNGPEPHGNKIEQPEIRADERQDAGGPANGAPSTSGGAYSQGAKSGGQAAPDPSGSYGIKDAPVAPATIAFEFDGQQVEAQPGETIWAVAKRLGTHIPHLCHKPDPGYRPDGNCRACMVEIEGERVLAASCKRTPAIGMKVKSATERATKARAMVLELLVADQPERATSHDPSSHFWVQADVLDVTESRFPAAERWTSDVSHPAMSVNLDACIQCNLCVRACREVQVNDVIGMAYRAAGSKVVFDFDDPMGGSTCVACGECVQACPTGALMPAAYLDANQTRTVYPDREVKSLCPYCGVGCQVSYKVKDERIVYAEGVNGPANQNRLCVKGRFGFDYVHHPHRLTVPLIRLENVPKDANDQVDPANPWTHFREATWEEALDRAAGGLKAIRDTNGRKALAGFGSAKGSNEEAYLFQKLVRLGFGTNNVDHCTRLCHASSVAALMEGLNSGAVTAPFSAALDAEVIVVIGANPTVNHPVAATFLKNAVKQRGAKLIIMDPRRQTLSRHAYRHLAFRPGSDVAMLNAMLNVIVTEGLYDEQYIAGYTENFEALREKIVDFTPEKMASVCGIDAETLREVARLYARAKSSLIFWGMGVSQHVHGTDNSRCLIALALITGQIGRPGTGLHPLRGQNNVQGASDAGLIPMVYPDYQSVEKDAVRELFEEFWGQSLDPQKGLTVVEIMRAIHAGEIRGMFVEGENPAMSDPDLNHARHALAMLDHLVVQDLFLTETAFHADVVLPASAFAEKAGTFTNTDRRVQIAQPVVAPPGDARQDWWIIQELARRLDLDWNYGGPADIFAEMAQVMPSLNNITWERLEREGAVTYPVDAPDQPGNEIIFYAGFPTESGRAKIVPAAIVPPDEVPDDEFPMVLSTGRVLEHWHTGSMTRRAGVLDALEPEAVAFMAPKELYRLGLRPGGSMRLETRRGAVVLKVRSDRDVPIGMIFMPFCYAEAAANLLTNPALDPLGKIPEFKFCAARVVPAEAAPMAAE

SEQ ID NO: 5 - protein, 572 aa, Methylobacterium extorquens
MSEASGTVRSFAHPGRGRNVARAVPKGRQVDPHAKVEIEELLGTRPRQRDLLIEHLHLIQDTYGQISADHLAALADEMSLAFAEVFETATFYAHFDVVKEGEADIPRLTIRVCDSITCAMFGADELLETLQRELASDAVRVVRAPCVGLCDHAPAVEVGHNFLHRADLASVRAAVEAEDTHAHIPTYVDYDAYRAGGGYATLERLRSGELPVDDVLKVLDDGGLRGLGGAGFPTGRKWRSVRGEPGPRLMAVNGDEGEPGTFKDQLYLNTDPHRFLEGMLIGAHVVEAADVYIYLRDEYPISREILAREIAKLPEGGTRIHLRRGAGAYICGEESSLIESLEGKRGLPRHKPPFPFQVGLFNRPTLINNIETLFWVRDLIERGAEWWKSHGRNGRVGLRSYSVSGRVKEPGVKLAPAGLTIQELIDEYCGGISDGHSFAAYLPGGASGGILPASMNDIPLDFGTLEKYGCFIGSAAVVILSDQDDVRGAALNLMKFFEDESCGQCTPCRSGTQKARMLMENGVWDTDLLGELAQCMRDASICGLGQAASNPVSTVIKYFPDLFPEPRAVAAE

SEQ ID NO: 6 - protein, 401 aa, Thiobacillus sp.
MAKILCVLYDDPVDGYPKTYARDDLPKIDHYPGGQTLPTPKAIDFTPGQLLGSVSGELGLRKYLEANGHTFVVTSDKDGPDSVFEKELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 7 - protein, 958 aa, Rhodobacter capsulatus
MKDLIIPPLDWTQDMGTPKREGAPVHLTIDGVEVTVPAGTSVLRAAAEAGISIPKLCATDNVEPVGSCRLCMVEIEGMRGTPTSCTTPVAPGMRVHTQTPQLQKLRRGVMELYISDHPLDCLTCAANGDCELQDMAGAVGLREVRYQAKDTHFARRDATGPNPRYIPKDNSNPYFSYDPAKCIVCMRCVRACEEVQGTFALTVMGRGFDARISPAAPDFLSSDCVSCGACVQACPTATLVEKSVERIGTPERKVVTTCAYCGVGCSFEAHMLGDQLVRMVPWKGGAANRGHSCVKGRFAYGYATHQDRILKPMIRDKITDPWREVNWTEALDFTATRLRALRDSHGADALGVITSSRCTNEETYLVQKLARAVFGTNNTDTCARVCHSPTGYGLKQTFGTSAGTQDFDSVEETDLALVIGANPTDGHPVFASRLRKRLRAGAKLIVVDPRRIDLLNTPHRGEAWHLQLKPGTNVAVMTAMAHVIVTEQIFDKRFIGDRCDWDEWADYAEFVANPEYAPEAVESLTGVPAGLLRQAARAYAAAPNAAIYYGLGVTEHSQGSTTVIAIANLAMMTGNIGRPGVGVNPLRGQNNVQGSCDMGSFPHEFPGYRHVSDDATRGLFERTWGVTLSSEPGLRIPNMLDAAVEGRFKALYVQGEDILQSDPDTRHVSAGLAAMDLVIVHDLFLNETANYAHVFLPGSTFLEKDGTFTNAERRINRVRRVMAPKAGFADWEVTQMLANALGAGWHYTHPSEIMAEIAATTPGFAAVTYEMLDARGSVQWPCNEKAPEGSPIMHVEGFVRGKGRFIRTAYLPTDEKTGPRFPLLLTTGRILSQYNVGAQTRRTENTVWHGEDRLEIHPTDAETRGIRDGDWVRLASRAGETTLRATVTDRVSPGVVYTTFHHPDTQANVVTTDTSDWATNCPEYKVTAVQVAASNGPSDWQQDYAAQAAAARRIEAAE

SEQ ID NO: 8 - protein, 500 aa, Rhodobacter capsulatus
MKIWLPCDAAAKACGAEAVLAALRLEAEKRGGALDIARNGSRGMIWLEPLLEVETPAGRIGFGPMTPADVPALFDALESHPKALGLVEEIPFFKRQTRLTFARCGRIEPLSLAQFAAAEGWAGLRKALKMTPAEVVEEVLASGLRGRGGAGFPTGIKWRTVAAAQADQKYIVCNVDEGDSGSFADRMLIEGDPFCLVEGMAIAGHAVGATRGYVYIRSEYPDAIAVMRAAIAMAKPFLAEAGFEMEVRVGAGAYVCGEETSLLNSLEGKRGTVRAKPPLPALKGLFGKPTVVNNLLSLAAVPWIIAHGAKAYESFGMDRSRGTIPLQIGGNVKRGGLFETGFGITLGELVEDICGGTASGRPVKAVQVGGPLGAYHPVSDYHLPFCYEQFAGQGGLVGHAGLVVHDDTADMLKLARFAMEFCAIESCGTCTPCRIGAVRGVEVIDRIAAGDASAMPLLDDLCQTMKLGSLCALGGFTPYPVQSAIRHFPADFPCAREAAE

SEQ ID NO: 9 - protein, 150 aa, Rhodobacter capsulatus
MTDTARLRAILAAHRGREGALLPILHDVQAAFGFIPEDAYAPIAADLGLTRAEVAGVVGFYHDFRKAPAGRHVIKLCRAEACQAMGMDAVQARLESALGLRLGDSSEAVTLEAVYCLGLCACAPAAMVDDRLVGRLDAAAVAGIVAELGA

SEQ ID NO: 10 - DNA, 5369 bp, Artificial Sequence
  pET-28a(+) vector
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 11 - DNA, 1911 bp, Unknown
  Carbon monoxide dehydrogenase II derived from Carboxydothermus hydrogenoformans
atggcgaaacaaaatctgaagagcaccgaccgtgcggttcaacaaatgctggataaagcgaagcgtgagggtattcaaaccgtgtgggatcgttacgaggcgatgaagccgcagtgcggtttcggcgaaaccggtctgtgctgccgtcactgcctgcaaggtccgtgccgtattaacccgtttggcgatgagccgaaagtgggcatttgcggtgcgaccgcggaagtgatcgttgcgcgtggtctggaccgtagcattgcggcgggtgcggcgggtcatagcggtcatgcgaagcacctggcgcacaccctgaagaaagcggtgcagggcaaagcggcgagctatatgattaaggaccgtaccaaactgcacagcatcgcgaagcgtctgggtattccgaccgaaggccaaaaagacgaggatattgcgctggaagttgcgaaagcggcgctggcggacttccatgagaaagataccccggttctgtgggtgaccaccgttctgccgccgagccgtgtgaaggttctgagcgcgcatggtctgatcccggcgggtattgatcacgaaatcgcggagattatgcaccgtaccagcatgggttgcgacgcggatgcgcagaacctgctgctgggtggcctgcgttgcagcctggcggacctggcgggttgctacatgggcaccgacctggcggatatcctgtttggtaccccggcgccggtggttaccgaaagcaacctgggcgtgctgaaggcggatgcggtgaacgttgcggtgcacggtcacaacccggttctgagcgacatcattgttagcgtgagcaaagagatggaaaacgaggcgcgtgcggcgggtgcgaccggtatcaacgtggttggtatttgctgcaccggcaacgaggtgctgatgcgtcacggtattccggcgtgcacccacagcgttagccaggaaatggcgatgatcaccggcgcgctggacgcgatgatcctggattatcagtgcattcaaccgagcgtggcgaccattgcggagtgcaccggtaccaccgttattaccaccatggaaatgagcaaaatcaccggtgcgacccatgtgaactttgcggaggaagcggcggttgagaacgcgaagcaaatcctgcgtctggcgattgatacctttaaacgtcgtaagggtaaaccggtggagatcccgaacattaagaccaaagtggttgcgggcttcagcaccgaagcgatcattaacgcgctgagcaagctgaacgcgaacgatccgctgaaaccgctgattgacaacgtggttaacggtaacatccgtggcgtgtgcctgttcgcgggttgcaacaacgttaaggtgccgcaggaccaaaactttaccaccattgcgcgtaagctgctgaaacagaacgttctggtggttgcgaccggttgcggtgcgggtgcgctgatgcgtcacggttttatggacccggcgaacgtggatgagctgtgcggcgacggtctgaaagcggttctgaccgcgatcggtgaagcgaacggtctgggtggcccgctgccgccggtgctgcacatgggtagctgcgttgacaacagccgtgcggtggcgctggttgcggcgctggcgaaccgtctgggcgttgacctggatcgtctgccggtggttgcgagcgcggcggaagcgatgcatgagaaggcggtggcgattggtacctgggcggttaccatcggtctgccgacccacattggtgtgctgccgccgatcaccggcagcctgccggtgacccaaatcctgaccagcagcgttaaagatattaccggtggctacttcatcgttgaactggacccggagaccgcggcggacaaactgctggcggcgatcaatgagcgtcgtgcgggtctgggtctgccgtggtga

SEQ ID NO: 12 - DNA, 1902 bp, Unknown
  Carbon monoxide dehydrogenase IV derived from Carboxydothermus hydrogenoformans
atggataaaagcaagttatcagtagatcctgtgatcccgaatttatatcgtaaagccagagaggaagggatttccactgtttttgatcgttatgaagcacagcagcctcagtgcggatttgggcttacaggcctttgctgtcgtcattgcgtgcagggaccgtgccgcatagatccgtttggggaaggaccgcaagcgggcatttgcggtgctaccgccgaggtaataacggctcgcaatctgctgcgtcaagtcacggcgggtgctgccgcccatgtagaccatgcgtatgatgtgctggaagttttggaacaaatcgcccaaggtacggaatcatacagtatcaaagatcaagaaaagttaaagcaggtcgcttttaccttaggtatagataccgctaacaaaacagagcaggagattgttgaagagatgtgccaaattatctatcgggattttgccaattctggtgcaacaccgatgacctatctgaaagccaactctcctcgggaacgccttgagacatgggaaaaattaggggttctgcctcgtaacccagaccgtgaaatcagagaagccttacaccagactacaatggggatggatgcggacccagtaaatttaatcttaaaaactattcgccttggtctggttgatggttttgcgggtctcaagttagcgacggatttacaggacatcatttttggcactccccagccagttgtcacagaagcaaatctgggtgtactcaaagaagattatgtgaatatcatagtccacggccatgttccgttacttagcgaaaaaattgttgaatggagcagaaagttggaagatgaggcgaaaaaagcaggggcaaaaggaatcaatcttgcaggtatatgttgtactggtaacgaggtgttaatgcgtcagggtgtaccgttagcaacaaacttcctggcccaggagttggcgattattacgggggcagttgacttaatggtagtggatgttcagtgtattatgccctcattagcagaaattgctgcttgctatcatacccgcttagtcactaccatgccgattgtgaaaattccaggtgcggagcatgttccgttcaccacagaaactgccgatgaggcctcgcagcagattgtgcgtatggctatagagagttaccaaaaacgcaatccagccaaggtctacatccccagggaaaaagccaaagttgtagcaggattcagcgttgaggcaatagtaaaagcgttagcgaaactaaatcctgacgatccgttaaagcccttaattgataatattgtgtctggcaacattctgggtgtggtcgccaccgttggttgtaacaatgtgaaggtcaagcatgattggttccacatagaattagtaaaagaactaattaaaaacaatgtgttagtggtgactacaggctgttccgctcatgcgttagcgaaagcaggcttgatggaccccgcagcagccgaatgggctggcgagggtttacgagccgtcttaacggcaataggcacggctaatgatttgggcggcccgctgccgcctgttttgcacatgggatcgtgtgttgataactcccgaatcggggacttagtgatcgccgtcgcaaactaccttaaagtaagtccaaaagaccttccgattgcggcttctgcacctgaataccagcatgaaaaggctttgagcatcggaacctgggcggtagcgatgggtattatgacccacttgggagttgttccacctgtggtcggaagttcaaaagttacccgtattcttacccaagatgccgaggctttaataggcggcaaattttatgtggaaacggacccatataaagcagcagcgggcatcattgaacatattaaggctaaacgggctctactgaatttataa

SEQ ID NO: 13 - DNA, 1890 bp, Thermococcus sp.
atggccggaaagaaggttccctcaaagcaagtctccataactccaggtgttggaaagcttattgagaaagccgaggaggatggggtcaagactgcctggcacagatttttggagcagcagcctcagtgtggattcggtctcttaggtgtctgctgtaagaactgtacaatgggaccatgtagaatcgatccgtttggggttggcccaactaagggagtttgtggtgcggatgcagatacaatagtagcaaggaacattgtaagaatgatagcggctggtactgccggtcacagcgatcactcaagagatgtagtccatgtattcaagggcattgctgaaggaaagttcaaggactataaactaacagatgttgaaaagctcaaagagctggctaagattctgggtgtcgaaacagagggcaagagcgaaaatgaaattgcattggaagtcgcccacattcttgagatggagttcggaaaacaggatgaggagccagtaagattacttgcagcaacagcaccaaagaagaggattaaggtctgggagaagctaggagtcttaccaagagccatcgacagggagatatgtctcagtatgcacagaacccacataggctgtgatgcagaccctgcaagccttctactgcatggtgtgaggactgccctggccgacggctggtgcggctcaatgatggccacttatctgagcgacattctctttggaacaccaaagccgataaagtcgctggcgaacctgggagtcttgaaggaagacatggtcaacataatcgttcacggccacaacccgattctctccatgaaaatagcagagattgcccagagtgaagagatgcagaagcttgcagagcagtacggagcaaagggaattaacgttgctggaatgtgctgtaccggaaacgaagttctctcaagaatgggagttcaggtcgctggaaacttcctaatgcaagagctggcgattataactggtgcagttgaggccgtgatagttgactaccagtgcctaatgccctcattagttgatgtcgcttcatgttaccacactaagataataactactgagccaaaggctcgcattccgggagcaatacacgtcgaatttgaacctgagaaagcggacgagatcgccaaagagatcatcaagattgcaattgagaactataagaacagagttccggcaaaagtctacattccagagcacaagatggaattggttgctggatttagtgtcgaggcaatacttgaagcccttggtggaacactggagcccctcataaaagccctccaggacggaacaataaagggaatcgtcggaatcgttggatgtaacaatccaagggtcaagcagaactacggtcacgtcaccttggccaaggagctcatcaagagggacatcctggttgttggaactggttgctggggaattgctgcagcaatgcatggattactaacccccgaagcagctgaaatggccggtccagggctgaaggcagtatgcgaagcgctcggaattccaccatgcctgcacatgggaagctgtgttgactgttcgagaatcctgctggtcttgagtgcccttgccaatgctctgaatgttgacatttcagacttgccagttgctggctctgctccagaatggatgagcgagaaggcagtggcaataggaacctacttcgttgcaagcggcgtcttcacgcacttgggagttatcccaccagtccttggaagccagaaggttaccaaactccttacggatgacatcgaggatctccttggagggaagttctacgttgagacagatccagtgaaagcggcagaaacaatatacaacgtgataattgagaagaggaaaaaacttggattgcccatc

SEQ ID NO: 14 - DNA, 7244 bp, Artificial Sequence
  plasmid comprising ChCODH II and His-tag
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggcgaaacaaaatctgaagagcaccgaccgtgcggttcaacaaatgctggataaagcgaagcgtgagggtattcaaaccgtgtgggatcgttacgaggcgatgaagccgcagtgcggtttcggcgaaaccggtctgtgctgccgtcactgcctgcaaggtccgtgccgtattaacccgtttggcgatgagccgaaagtgggcatttgcggtgcgaccgcggaagtgatcgttgcgcgtggtctggaccgtagcattgcggcgggtgcggcgggtcatagcggtcatgcgaagcacctggcgcacaccctgaagaaagcggtgcagggcaaagcggcgagctatatgattaaggaccgtaccaaactgcacagcatcgcgaagcgtctgggtattccgaccgaaggccaaaaagacgaggatattgcgctggaagttgcgaaagcggcgctggcggacttccatgagaaagataccccggttctgtgggtgaccaccgttctgccgccgagccgtgtgaaggttctgagcgcgcatggtctgatcccggcgggtattgatcacgaaatcgcggagattatgcaccgtaccagcatgggttgcgacgcggatgcgcagaacctgctgctgggtggcctgcgttgcagcctggcggacctggcgggttgctacatgggcaccgacctggcggatatcctgtttggtaccccggcgccggtggttaccgaaagcaacctgggcgtgctgaaggcggatgcggtgaacgttgcggtgcacggtcacaacccggttctgagcgacatcattgttagcgtgagcaaagagatggaaaacgaggcgcgtgcggcgggtgcgaccggtatcaacgtggttggtatttgctgcaccggcaacgaggtgctgatgcgtcacggtattccggcgtgcacccacagcgttagccaggaaatggcgatgatcaccggcgcgctggacgcgatgatcctggattatcagtgcattcaaccgagcgtggcgaccattgcggagtgcaccggtaccaccgttattaccaccatggaaatgagcaaaatcaccggtgcgacccatgtgaactttgcggaggaagcggcggttgagaacgcgaagcaaatcctgcgtctggcgattgatacctttaaacgtcgtaagggtaaaccggtggagatcccgaacattaagaccaaagtggttgcgggcttcagcaccgaagcgatcattaacgcgctgagcaagctgaacgcgaacgatccgctgaaaccgctgattgacaacgtggttaacggtaacatccgtggcgtgtgcctgttcgcgggttgcaacaacgttaaggtgccgcaggaccaaaactttaccaccattgcgcgtaagctgctgaaacagaacgttctggtggttgcgaccggttgcggtgcgggtgcgctgatgcgtcacggttttatggacccggcgaacgtggatgagctgtgcggcgacggtctgaaagcggttctgaccgcgatcggtgaagcgaacggtctgggtggcccgctgccgccggtgctgcacatgggtagctgcgttgacaacagccgtgcggtggcgctggttgcggcgctggcgaaccgtctgggcgttgacctggatcgtctgccggtggttgcgagcgcggcggaagcgatgcatgagaaggcggtggcgattggtacctgggcggttaccatcggtctgccgacccacattggtgtgctgccgccgatcaccggcagcctgccggtgacccaaatcctgaccagcagcgttaaagatattaccggtggctacttcatcgttgaactggacccggagaccgcggcggacaaactgctggcggcgatcaatgagcgtcgtgcgggtctgggtctgccgtggtgaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 15 - DNA, 7195 bp, Artificial Sequence
  plasmid comprising ChCODH IV and His-tag
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttataaattcagtagagcccgtttagccttaatatgttcaatgatgcccgctgctgctttatatgggtccgtttccacataaaatttgccgcctattaaagcctcggcatcttgggtaagaatacgggtaacttttgaacttccgaccacaggtggaacaactcccaagtgggtcataatacccatcgctaccgcccaggttccgatgctcaaagccttttcatgctggtattcaggtgcagaagccgcaatcggaaggtcttttggacttactttaaggtagtttgcgacggcgatcactaagtccccgattcgggagttatcaacacacgatcccatgtgcaaaacaggcggcagcgggccgcccaaatcattagccgtgcctattgccgttaagacggctcgtaaaccctcgccagcccattcggctgctgcggggtccatcaagcctgctttcgctaacgcatgagcggaacagcctgtagtcaccactaacacattgtttttaattagttcttttactaattctatgtggaaccaatcatgcttgaccttcacattgttacaaccaacggtggcgaccacacccagaatgttgccagacacaatattatcaattaagggctttaacggatcgtcaggatttagtttcgctaacgcttttactattgcctcaacgctgaatcctgctacaactttggctttttccctggggatgtagaccttggctggattgcgtttttggtaactctctatagccatacgcacaatctgctgcgaggcctcatcggcagtttctgtggtgaacggaacatgctccgcacctggaattttcacaatcggcatggtagtgactaagcgggtatgatagcaagcagcaatttctgctaatgagggcataatacactgaacatccactaccattaagtcaactgcccccgtaataatcgccaactcctgggccaggaagtttgttgctaacggtacaccctgacgcattaacacctcgttaccagtacaacatatacctgcaagattgattccttttgcccctgcttttttcgcctcatcttccaactttctgctccattcaacaattttttcgctaagtaacggaacatggccgtggactatgatattcacataatcttctttgagtacacccagatttgcttctgtgacaactggctggggagtgccaaaaatgatgtcctgtaaatccgtcgctaacttgagacccgcaaaaccatcaaccagaccaaggcgaatagtttttaagattaaatttactgggtccgcatccatccccattgtagtctggtgtaaggcttctctgatttcacggtctgggttacgaggcagaacccctaatttttcccatgtctcaaggcgttcccgaggagagttggctttcagataggtcatcggtgttgcaccagaattggcaaaatcccgatagataatttggcacatctcttcaacaatctcctgctctgttttgttagcggtatctatacctaaggtaaaagcgacctgctttaacttttcttgatctttgatactgtatgattccgtaccttgggcgatttgttccaaaacttccagcacatcatacgcatggtctacatgggcggcagcacccgccgtgacttgacgcagcagattgcgagccgttattacctcggcggtagcaccgcaaatgcccgcttgcggtccttccccaaacggatctatgcggcacggtccctgcacgcaatgacgacagcaaaggcctgtaagcccaaatccgcactgaggctgctgtgcttcataacgatcaaaaacagtggaaatcccttcctctctggctttacgatataaattcgggatcacaggatctactgataacttgcttttatccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 16 - DNA, 6749 bp, Artificial Sequence
  plasmid comprising ToCODH and His-tag
gtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatggccggaaagaaggttccctcaaagcaagtctccataactccaggtgttggaaagcttattgagaaagccgaggaggatggggtcaagactgcctggcacagatttttggagcagcagcctcagtgtggattcggtctcttaggtgtctgctgtaagaactgtacaatgggaccatgtagaatcgatccgtttggggttggcccaactaagggagtttgtggtgcggatgcagatacaatagtagcaaggaacattgtaagaatgatagcggctggtactgccggtcacagcgatcactcaagagatgtagtccatgtattcaagggcattgctgaaggaaagttcaaggactataaactaacagatgttgaaaagctcaaagagctggctaagattctgggtgtcgaaacagagggcaagagcgaaaatgaaattgcattggaagtcgcccacattcttgagatggagttcggaaaacaggatgaggagccagtaagattacttgcagcaacagcaccaaagaagaggattaaggtctgggagaagctaggagtcttaccaagagccatcgacagggagatatgtctcagtatgcacagaacccacataggctgtgatgcagaccctgcaagccttctactgcatggtgtgaggactgccctggccgacggctggtgcggctcaatgatggccacttatctgagcgacattctctttggaacaccaaagccgataaagtcgctggcgaacctgggagtcttgaaggaagacatggtcaacataatcgttcacggccacaacccgattctctccatgaaaatagcagagattgcccagagtgaagagatgcagaagcttgcagagcagtacggagcaaagggaattaacgttgctggaatgtgctgtaccggaaacgaagttctctcaagaatgggagttcaggtcgctggaaacttcctaatgcaagagctggcgattataactggtgcagttgaggccgtgatagttgactaccagtgcctaatgccctcattagttgatgtcgcttcatgttaccacactaagataataactactgagccaaaggctcgcattccgggagcaatacacgtcgaatttgaacctgagaaagcggacgagatcgccaaagagatcatcaagattgcaattgagaactataagaacagagttccggcaaaagtctacattccagagcacaagatggaattggttgctggatttagtgtcgaggcaatacttgaagcccttggtggaacactggagcccctcataaaagccctccaggacggaacaataaagggaatcgtcggaatcgttggatgtaacaatccaagggtcaagcagaactacggtcacgtcaccttggccaaggagctcatcaagagggacatcctggttgttggaactggttgctggggaattgctgcagcaatgcatggattactaacccccgaagcagctgaaatggccggtccagggctgaaggcagtatgcgaagcgctcggaattccaccatgcctgcacatgggaagctgtgttgactgttcgagaatcctgctggtcttgagtgcccttgccaatgctctgaatgttgacatttcagacttgccagttgctggctctgctccagaatggatgagcgagaaggcagtggcaataggaacctacttcgttgcaagcggcgtcttcacgcacttgggagttatcccaccagtccttggaagccagaaggttaccaaactccttacggatgacatcgaggatctccttggagggaagttctacgttgagacagatccagtgaaagcggcagaaacaatatacaacgtgataattgagaagaggaaaaaacttggattgcccatccatcaccatcatccccactaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaa

SEQ ID NO: 17 - DNA, 2967 bp, Methylobacterium extorquens
atgagcaacggccccgaaccgcacggcaacaagatcgaacagcccgagatccgcgccgacgaacgtcaggatgccggcgggccggcaaatggcgcaccatcgacctccggcggcgcctactcgcagggcgccaagtcgggtggccaggccgcgccggatccgtccggctcctacggcatcaaggacgccccggtcgcgcccgcgaccatcgccttcgagttcgacggccaacaggtcgaggcccagcccggcgagacgatctgggcggtcgccaagcgccttggcacgcatatcccgcatctctgccacaagcccgatcccggctaccgcccggacggcaattgccgcgcctgcatggtcgagatcgagggcgagcgcgtgctcgccgcctcgtgcaagcgcacgcccgccatcggcatgaaggtgaagtcggccaccgagcgcgccaccaaggcccgcgccatggtgctcgaactgctcgtggccgatcagcccgagcgcgcgacctcgcatgacccgtcgtcgcatttctgggtgcaggccgacgtcctcgatgtgaccgagagccgcttcccggcggccgagcgctggaccagcgacgtcagccacccggcgatgagcgtcaatctcgacgcctgcatccagtgcaatctctgtgtccgcgcctgccgcgaggttcaggtcaacgacgtgatcggcatggcctaccgcgccgcgggctccaaggtcgtgtttgacttcgacgatccgatgggtggctccacctgcgtggcctgcggcgagtgcgtccaagcctgcccgaccggggcgctgatgccggccgcctatctcgacgcaaaccagacccggacggtctatcccgaccgcgaggtgaagtcgctctgcccctattgcggcgtcggctgccaagtctcctacaaggtcaaggacgagcgcatcgtctacgccgagggcgtgaacggaccggccaaccagaaccggctctgcgtgaagggccgcttcggcttcgactacgtccaccacccccaccgcctgacggtgccgctgatccgcttggagaacgtgcccaaggacgccaacgatcaggtcgatccggcgaacccctggacgcatttccgcgaggcgacctgggaagaggcgctcgaccgcgcggcgggcggcctgaaggcgatccgtgacaccaacgggcgcaaggcgctggcgggcttcggctcggccaagggttcgaacgaggaggcctacctcttccagaagctcgtccgcctcggcttcggcaccaacaacgtcgatcactgcacgcgcctgtgccacgcctcgtcggtggcggcgctgatggagggcctgaattccggcgccgtcaccgctcccttctcggcagcgctcgacgccgaggtcatcgtcgtcatcggcgccaacccgaccgtgaaccatccggtcgcggcgaccttcctcaagaacgcggtgaagcagcgcggcgccaagctgatcatcatggacccgcggcgccagacgctctcgcgccacgcctatcggcacctcgccttccgccccggctcggacgtggcgatgctcaacgcgatgctcaacgtgatcgtcacggagggcctctacgacgagcagtacatcgccggctacaccgagaacttcgaggctctgcgcgagaagatcgtcgacttcacgccggagaagatggcctcggtctgcggcatcgacgccgagaccctgcgcgaggtcgcccggctctacgcccgggccaagtcgtcgctcatcttctggggcatgggcgtcagccagcacgtgcacggcaccgacaactcgcgctgcctgatcgcgctcgccctcatcaccggccagatcggccggcccggcaccggcctgcacccgttgcgcggccagaacaacgtccagggcgcgtccgatgccggcctgatcccgatggtctacccggactatcagtcggtcgagaaggacgcggtgcgtgagctgttcgaggagttctgggggcagtccctcgatcctcagaagggcctcaccgtggtcgagatcatgcgcgcgatccacgcgggcgagatccggggcatgttcgtcgagggcgagaacccggcgatgtccgaccccgacctcaaccatgcccgccacgcgctggcgatgctcgaccatctcgtggtgcaggacctgttcctgacggagacggccttccacgccgacgtggtgctgccggcctcggcctttgccgagaaagccgggaccttcaccaacaccgaccggcgcgtgcagatcgcccagcccgtcgtcgcccctccgggcgatgcgcgccaggattggtggatcatccaggaactggcccgacgcctcgacctcgactggaactacggcggcccggccgacatcttcgccgagatggcgcaggtgatgccgtccttgaacaacatcacctgggagcggctggagcgcgagggggcggtgacctatccggtcgatgccccggaccagcccggcaacgagatcatcttctatgccggcttcccgaccgagagcggtcgcgccaagatcgtgcccgcggcgatcgtgccgccggacgaggtgccggacgacgagttcccgatggtgctctcgaccggccgcgtgctcgaacactggcacacgggctcgatgacccggcgcgcgggcgtgctcgacgcgctggagccggaggcggtggccttcatggcacccaaggagctctaccggctcggtctccggcccggcgggtcgatgcggttggaaacacggcgcggcgccgtcgtgttgaaggtgcgctccgaccgggacgtgccgatcggcatgatcttcatgcccttctgctacgcggaagccgccgccaaccttctgaccaaccccgccctcgaccccctcggaaagattcccgagttcaaattctgcgcagcccgcgtcgtccccgcggaggctgcgccgatggccgccgag

SEQ ID NO: 18 - DNA, 1719 bp, Methylobacterium extorquens
atgagtgaggcaagtgggactgtccggagcttcgcgcatccgggccgtggccgtaacgtcgcccgcgccgtgccgaaggggcgtcaggtcgatccccacgccaaggtcgagatcgaggagctgctcggcacccgcccgcgccagcgcgacctgctgatcgagcacctgcacctgatccaagacacctacggccagatcagcgccgatcatctcgcggcgctggccgacgagatgagcctcgccttcgccgaggtgttcgagaccgcgaccttctacgcgcatttcgatgtggtgaaggagggcgaggccgacatcccgcgcctgacgatccgggtttgcgacagcatcacctgcgccatgttcggcgccgacgagctgctggagacgctgcagcgcgagctggcctcggatgcggtccgcgtcgtgcgcgcgccctgtgtcggcctgtgcgaccacgccccggcggtcgaggtcgggcacaacttcctgcaccgggccgacctcgcctccgtgcgcgccgcggtcgaggccgaggacacccacgcccacatccccacttacgtcgattacgacgcctaccgggccggcggcggctacgcgaccctggagcggctgcgcagcggcgaactgccggtcgatgacgtgctgaaggtgctcgacgacggcggcctgcgcggcctcggcggcgccggctttcccaccggccgcaagtggcgctccgtgcgcggcgagcccggaccccggctgatggcggtcaacggcgacgagggcgagcccggcaccttcaaggatcagctctacctcaacaccgatccgcaccgctttctggaaggcatgctgatcggcgcccacgtcgtcgaggccgccgacgtctacatctacctgcgcgacgagtacccgatctcccgcgagatcctggcccgcgagatcgcgaagctccccgagggcggcacccgcatccacctgcgccgtggggccggcgcctatatctgcggcgaggaatcctcgctgatcgagtcgctggagggcaagcgcggcctgccgcggcacaagccgcccttccccttccaggtcggcctgttcaaccggccgacgctgatcaacaacatcgagacgctgttctgggtgcgcgacctgatcgagcgcggcgccgaatggtggaagagccacgggcgcaacggccgcgtcggcctgcgctcgtactcggtttcgggccgggtcaaggagccgggcgtcaagctcgcgcccgccggcctgaccatccaggaactcatcgacgagtattgcggcggcatctctgacggccacagcttcgcggcctacctgccgggcggagcctcgggcggcatcctgccggcctcgatgaacgacatcccgctcgatttcggcacgctggaaaaatacggctgcttcatcggctcggccgcggtcgtgatcctgtccgatcaggacgatgtgcgcggtgccgcgttgaacctgatgaagttcttcgaggacgagtcctgcgggcagtgcacgccctgccgctcgggcacgcagaaggcccgcatgctgatggagaacggcgtgtgggacaccgatctcctcggcgagctggcgcaatgcatgcgcgacgcctcgatctgcggtctcggtcaggcggcctcgaaccccgtcagcaccgtgatcaagtacttccctgatctcttcccggagccgcgggccgtggcggccgagtga

SEQ ID NO: 19 - DNA, 5834 bp, Artificial Sequence
  pCM110 vector
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgatccatatgtaagatttaaatgcaaccgttttttcggaaggaaatgatgacctcgtttccaccgaattagcttgcatgcctgcaggtcgactctagatcacctccttaagcttcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcggatcctaagggcgaattccagcacactggcggccgttactagtggatccgagctcggtaccatcgatagatccgttcagggcatcaaatgcagactcgtttctgacacttgcagactggatctatctcatctgcgcaaggcagaacgtgaagacggccgccctggacctcgcccgcgagcgccaggcgcacgaggccggcgcgcggacccgcgccacggcccacgagcggacgccgcagcaggagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 20 - DNA, 10907 bp, Artificial Sequence
  plasmid comprising MeFDH I and His-tag
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgatccatatgtaagatttaaatgcaaccgttttttcggaaggaaatgatgacctcgtttccaccgaattagcttgcatgcctgcaggtcgactctagatcacctccttaagcttcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcggatcctaagggcgaattccagcacactggcggccgttactagaaataattttgtttaactttaagaaggagatatacatatgagtgaggcaagtgggactgtccggagcttcgcgcatccgggccgtggccgtaacgtcgcccgcgccgtgccgaaggggcgtcaggtcgatccccacgccaaggtcgagatcgaggagctgctcggcacccgcccgcgccagcgcgacctgctgatcgagcacctgcacctgatccaagacacctacggccagatcagcgccgatcatctcgcggcgctggccgacgagatgagcctcgccttcgccgaggtgttcgagaccgcgaccttctacgcgcatttcgatgtggtgaaggagggcgaggccgacatcccgcgcctgacgatccgggtttgcgacagcatcacctgcgccatgttcggcgccgacgagctgctggagacgctgcagcgcgagctggcctcggatgcggtccgcgtcgtgcgcgcgccctgtgtcggcctgtgcgaccacgccccggcggtcgaggtcgggcacaacttcctgcaccgggccgacctcgcctccgtgcgcgccgcggtcgaggccgaggacacccacgcccacatccccacttacgtcgattacgacgcctaccgggccggcggcggctacgcgaccctggagcggctgcgcagcggcgaactgccggtcgatgacgtgctgaaggtgctcgacgacggcggcctgcgcggcctcggcggcgccggctttcccaccggccgcaagtggcgctccgtgcgcggcgagcccggaccccggctgatggcggtcaacggcgacgagggcgagcccggcaccttcaaggatcagctctacctcaacaccgatccgcaccgctttctggaaggcatgctgatcggcgcccacgtcgtcgaggccgccgacgtctacatctacctgcgcgacgagtacccgatctcccgcgagatcctggcccgcgagatcgcgaagctccccgagggcggcacccgcatccacctgcgccgtggggccggcgcctatatctgcggcgaggaatcctcgctgatcgagtcgctggagggcaagcgcggcctgccgcggcacaagccgcccttccccttccaggtcggcctgttcaaccggccgacgctgatcaacaacatcgagacgctgttctgggtgcgcgacctgatcgagcgcggcgccgaatggtggaagagccacgggcgcaacggccgcgtcggcctgcgctcgtactcggtttcgggccgggtcaaggagccgggcgtcaagctcgcgcccgccggcctgaccatccaggaactcatcgacgagtattgcggcggcatctctgacggccacagcttcgcggcctacctgccgggcggagcctcgggcggcatcctgccggcctcgatgaacgacatcccgctcgatttcggcacgctggaaaaatacggctgcttcatcggctcggccgcggtcgtgatcctgtccgatcaggacgatgtgcgcggtgccgcgttgaacctgatgaagttcttcgaggacgagtcctgcgggcagtgcacgccctgccgctcgggcacgcagaaggcccgcatgctgatggagaacggcgtgtgggacaccgatctcctcggcgagctggcgcaatgcatgcgcgacgcctcgatctgcggtctcggtcaggcggcctcgaaccccgtcagcaccgtgatcaagtacttccctgatctcttcccggagccgcgggccgtggcggccgagtgacgcccagcccgacgatgtctcctcacccgatgccagagcctggagcggacgcatgagcaacggccccgaaccgcacggcaacaagatcgaacagcccgagatccgcgccgacgaacgtcaggatgccggcgggccggcaaatggcgcaccatcgacctccggcggcgcctactcgcagggcgccaagtcgggtggccaggccgcgccggatccgtccggctcctacggcatcaaggacgccccggtcgcgcccgcgaccatcgccttcgagttcgacggccaacaggtcgaggcccagcccggcgagacgatctgggcggtcgccaagcgccttggcacgcatatcccgcatctctgccacaagcccgatcccggctaccgcccggacggcaattgccgcgcctgcatggtcgagatcgagggcgagcgcgtgctcgccgcctcgtgcaagcgcacgcccgccatcggcatgaaggtgaagtcggccaccgagcgcgccaccaaggcccgcgccatggtgctcgaactgctcgtggccgatcagcccgagcgcgcgacctcgcatgacccgtcgtcgcatttctgggtgcaggccgacgtcctcgatgtgaccgagagccgcttcccggcggccgagcgctggaccagcgacgtcagccacccggcgatgagcgtcaatctcgacgcctgcatccagtgcaatctctgtgtccgcgcctgccgcgaggttcaggtcaacgacgtgatcggcatggcctaccgcgccgcgggctccaaggtcgtgtttgacttcgacgatccgatgggtggctccacctgcgtggcctgcggcgagtgcgtccaagcctgcccgaccggggcgctgatgccggccgcctatctcgacgcaaaccagacccggacggtctatcccgaccgcgaggtgaagtcgctctgcccctattgcggcgtcggctgccaagtctcctacaaggtcaaggacgagcgcatcgtctacgccgagggcgtgaacggaccggccaaccagaaccggctctgcgtgaagggccgcttcggcttcgactacgtccaccacccccaccgcctgacggtgccgctgatccgcttggagaacgtgcccaaggacgccaacgatcaggtcgatccggcgaacccctggacgcatttccgcgaggcgacctgggaagaggcgctcgaccgcgcggcgggcggcctgaaggcgatccgtgacaccaacgggcgcaaggcgctggcgggcttcggctcggccaagggttcgaacgaggaggcctacctcttccagaagctcgtccgcctcggcttcggcaccaacaacgtcgatcactgcacgcgcctgtgccacgcctcgtcggtggcggcgctgatggagggcctgaattccggcgccgtcaccgctcccttctcggcagcgctcgacgccgaggtcatcgtcgtcatcggcgccaacccgaccgtgaaccatccggtcgcggcgaccttcctcaagaacgcggtgaagcagcgcggcgccaagctgatcatcatggacccgcggcgccagacgctctcgcgccacgcctatcggcacctcgccttccgccccggctcggacgtggcgatgctcaacgcgatgctcaacgtgatcgtcacggagggcctctacgacgagcagtacatcgccggctacaccgagaacttcgaggctctgcgcgagaagatcgtcgacttcacgccggagaagatggcctcggtctgcggcatcgacgccgagaccctgcgcgaggtcgcccggctctacgcccgggccaagtcgtcgctcatcttctggggcatgggcgtcagccagcacgtgcacggcaccgacaactcgcgctgcctgatcgcgctcgccctcatcaccggccagatcggccggcccggcaccggcctgcacccgttgcgcggccagaacaacgtccagggcgcgtccgatgccggcctgatcccgatggtctacccggactatcagtcggtcgagaaggacgcggtgcgtgagctgttcgaggagttctgggggcagtccctcgatcctcagaagggcctcaccgtggtcgagatcatgcgcgcgatccacgcgggcgagatccggggcatgttcgtcgagggcgagaacccggcgatgtccgaccccgacctcaaccatgcccgccacgcgctggcgatgctcgaccatctcgtggtgcaggacctgttcctgacggagacggccttccacgccgacgtggtgctgccggcctcggcctttgccgagaaagccgggaccttcaccaacaccgaccggcgcgtgcagatcgcccagcccgtcgtcgcccctccgggcgatgcgcgccaggattggtggatcatccaggaactggcccgacgcctcgacctcgactggaactacggcggcccggccgacatcttcgccgagatggcgcaggtgatgccgtccttgaacaacatcacctgggagcggctggagcgcgagggggcggtgacctatccggtcgatgccccggaccagcccggcaacgagatcatcttctatgccggcttcccgaccgagagcggtcgcgccaagatcgtgcccgcggcgatcgtgccgccggacgaggtgccggacgacgagttcccgatggtgctctcgaccggccgcgtgctcgaacactggcacacgggctcgatgacccggcgcgcgggcgtgctcgacgcgctggagccggaggcggtggccttcatggcacccaaggagctctaccggctcggtctccggcccggcgggtcgatgcggttggaaacacggcgcggcgccgtcgtgttgaaggtgcgctccgaccgggacgtgccgatcggcatgatcttcatgcccttctgctacgcggaagccgccgccaaccttctgaccaaccccgccctcgaccccctcggaaagattcccgagttcaaattctgcgcagcccgcgtcgtccccgcggaggctgcgccgatggccgccgagcaccaccaccaccaccactaaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatagatccgttcagggcatcaaatgcagactcgtttctgacacttgcagactggatctatctcatctgcgcaaggcagaacgtgaagacggccgccctggacctcgcccgcgagcgccaggcgcacgaggccggcgcgcggacccgcgccacggcccacgagcggacgccgcagcaggagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 21 - DNA, 1203 bp, Thiobacillus sp.
atggcgaaaatactttgcgttctctatgatgatccggtcgacggctatccgaagacctatgcgcgcgacgatctgccgaagatcgaccactatccgggcgggcagaccctgccgacgccgaaggcgatcgacttcacccccggccagcttctcggctcggtctccggcgagctgggcctgcgcaaatatctcgaagccaacggccacaccttcgtcgtcacctccgacaaggacggcccggattcggtgttcgagaaggagctcgtcgacgccgacgtggtgatctcccagcccttctggccggcctatctgacacctgagcgcatcgccaaggcgaagaacctgaagctggcgctcaccgccggcatcggctccgatcacgtcgatcttcagtcggcgatcgaccgcggcatcaccgtggccgaagtcacctactgcaactcgatcagcgtcgccgagcatgtggtgatgatgatcctcggcctggtgcgcaactacattccctcgcatgactgggcgcgcaagggcggctggaacatagccgactgcgtggagcactcctacgacctcgagggcatgaccgtcggctccgttgccgccggccgcatcggcctcgccgtgctgcgccgcctcgcgcccttcgacgtgaagctgcactacaccgaccgccaccgcctgccggaagcggtcgagaaggagttgggcctggtctggcacgacacccgcgaggacatgtacccacattgcgacgtggtcacgctcaacgtgccgctgcatcccgagacggagcacatgatcaatgacgagacgctgaagctgttcaagcgcggcgcctatatcgtcaacaccgcccgcggcaagctcgccgaccgcgacgccatcgtgcgtgcgatcgagagcgggcagctcgccggctatgctggcgacgtgtggttcccgcagccggcgccgaaggatcacccctggcgcaccatgaagtgggaaggcatgacgccgcacatttccggcacctcgctctccgcccaggcgcgctatgcggcgggcacgcgcgagatcctcgaatgcttcttcgaaggtcggccgatccgcgacgagtacctgatcgtgcagggcggcgcactcgccggcacgggggcgcattcctactccaagggcaatgcgaccggcggctcggaagaggccgcgaagttcaagaaggccggc

SEQ ID NO: 22 - DNA, 4857 bp, Artificial Sequence
  plasmid comprising TsFDH and His-tag
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcttaatgatggtggtgatgatggccggccttcttgaacttcgcggcctcttccgagccgccggtcgcattgcccttggagtaggaatgcgcccccgtgccggcgagtgcgccgccctgcacgatcaggtactcgtcgcggatcggccgaccttcgaagaagcattcgaggatctcgcgcgtgcccgccgcatagcgcgcctgggcggagagcgaggtgccggaaatgtgcggcgtcatgccttcccacttcatggtgcgccaggggtgatccttcggcgccggctgcgggaaccacacgtcgccagcatagccggcgagctgcccgctctcgatcgcacgcacgatggcgtcgcggtcggcgagcttgccgcgggcggtgttgacgatataggcgccgcgcttgaacagcttcagcgtctcgtcattgatcatgtgctccgtctcgggatgcagcggcacgttgagcgtgaccacgtcgcaatgtgggtacatgtcctcgcgggtgtcgtgccagaccaggcccaactccttctcgaccgcttccggcaggcggtggcggtcggtgtagtgcagcttcacgtcgaagggcgcgaggcggcgcagcacggcgaggccgatgcggccggcggcaacggagccgacggtcatgccctcgaggtcgtaggagtgctccacgcagtcggctatgttccagccgcccttgcgcgcccagtcatgcgagggaatgtagttgcgcaccaggccgaggatcatcatcaccacatgctcggcgacgctgatcgagttgcagtaggtgacttcggccacggtgatgccgcggtcgatcgccgactgaagatcgacgtgatcggagccgatgccggcggtgagcgccagcttcaggttcttcgccttggcgatgcgctcaggtgtcagataggccggccagaagggctgggagatcaccacgtcggcgtcgacgagctccttctcgaacaccgaatccgggccgtccttgtcggaggtgacgacgaaggtgtggccgttggcttcgagatatttgcgcaggcccagctcgccggagaccgagccgagaagctggccgggggtgaagtcgatcgccttcggcgtcggcagggtctgcccgcccggatagtggtcgatcttcggcagatcgtcgcgcgcataggtcttcggatagccgtcgaccggatcatcatagagaacgcaaagtattttcgccatgctagccatatgtatatctccttcttaaagttaaacaaaattatttctagagggaaaccgttgtggtctccctatagtgagtcgtattaatttcgcgggatcgagatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 23 - DNA, 2877 bp, Rhodobacter capsulatus
atgaaagacctgattatcccgccgctggactggacgcaagacatgggtacgccgaaacgcgaaggtgctccggtgcatctgacgattgatggcgtcgaagtgacggttccggcgggtaccagcgttctgcgtgccgcagcagaagctggcatttctatcccgaaactgtgcgcgaccgataacgtggaaccggttggtagttgccgtctgtgtatggtggaaattgaaggcatgcgtggtaccccgacgtcctgtaccacgccggttgccccgggtatgcgtgtccatacccagacgccgcagctgcaaaaactgcgtcgcggtgtgatggaactgtatatcagcgatcacccgctggactgcctgacctgtgctgcgaacggcgattgcgaactgcaggacatggcaggcgctgtgggtctgcgtgaagttcgctatcaagcgaaagatacgcattttgcgcgtcgcgatgccaccggtccgaatccgcgttacattccgaaagataactcaaatccgtatttctcgtacgacccggccaaatgcatcgtctgtatgcgttgcgtgcgcgcctgcgaagaagtgcagggcacgtttgcactgaccgttatgggccgtggtttcgatgcgcgcattagtccggccgcaccggattttctgagcagcgactgtgtgtcctgcggcgcctgtgttcaggcatgtccgaccgctaccctggtggaaaaatctgtggaacgtatcggtaccccggaacgcaaagtggttaccacgtgcgcctattgtggcgttggttgctcgtttgaagcacatatgctgggcgatcagctggtccgtatggtgccgtggaaaggcggtgctgcgaaccgtggtcatagctgtgtgaaaggccgcttcgcgtatggttacgccacccaccaggatcgtattctgaaaccgatgattcgcgataaaatcacggacccgtggcgcgaagttaactggaccgaagcactggattttaccgctacgcgtctgcgcgcgctgcgtgatagccatggtgccgatgcactgggtgtgatcaccagttcccgctgcacgaatgaagaaacctatctggttcagaaactggcacgtgctgtcttcggtacgaacaataccgatacgtgcgcccgcgtttgtcattctccgaccggctacggtctgaaacagacctttggcacgagtgccggtacgcaagatttcgactccgtcgaagaaaccgatctggccctggtgattggcgcaaatccgaccgacggtcacccggtgtttgcgagtcgtctgcgtaaacgtctgcgtgccggtgcaaaactgattgtcgtggatccgcgtcgcatcgacctgctgaacaccccgcatcgtggcgaagcatggcacctgcagctgaaaccgggtacgaatgtcgcagtgatgaccgcaatggctcatgttattgtcaccgaacaaatctttgataaacgtttcatcggtgaccgctgcgattgggacgaatgggcggattatgccgaattcgtggcgaacccggaatacgcaccggaagctgtcgaaagcctgaccggcgtgccggctggtctgctgcgtcaggcggcacgtgcctatgctgccgcaccgaatgcagctatttattacggcctgggtgtgaccgaacacagccaaggctctaccacggttattgcgatcgccaacctggccatgatgaccggcaatatcggtcgtccgggcgtgggtgttaacccgctgcgtggtcagaacaatgtgcaaggcagctgtgatatgggttcttttccgcatgaattcccgggttatcgccacgttagtgatgacgcgacccgtggcctgtttgaacgcacgtggggtgtgaccctgtcatcggaaccgggcctgcgtattccgaatatgctggatgcggccgttgaaggccgcttcaaagcgctgtatgtccagggtgaagacattctgcaatcagatccggacacccgtcatgtttcggccggtctggcagctatggatctggtcatcgtgcatgacctgtttctgaacgaaacggcgaattacgcccacgtgtttctgccgggctccaccttcctggaaaaagatggtacctttacgaacgcggaacgtcgcattaatcgcgttcgtcgcgtcatggcaccgaaagctggtttcgcggattgggaagtgacgcagatgctggcaaacgctctgggcgctggttggcattatacccacccgtcagaaattatggcggaaattgccgcaaccacgccgggttttgcagctgtcacctacgaaatgctggatgcacgtggttcagtgcagtggccgtgcaatgaaaaagcgccggaaggctcgccgattatgcatgtcgaaggttttgtgcgcggcaaaggtcgtttcatccgcaccgcgtatctgccgacggatgaaaaaaccggtccgcgtttcccgctgctgctgaccacgggccgcattctgagccagtacaacgttggtgcccaaacccgtcgcacggaaaataccgtctggcatggtgaagatcgcctggaaattcacccgacggacgcagaaacccgtggcatccgcgatggtgattgggttcgtctggccagccgtgcaggtgaaaccaccctgcgtgcaaccgttacggatcgcgtctctccgggtgttgtctataccacgtttcatcacccggacacccaggcgaacgtggttaccacggatacgtctgactgggctaccaattgtccggaatacaaagtgaccgcggttcaagtcgcggccagtaatggcccgtccgattggcaacaggactatgcggcacaagcagcagcagcccgtcgtattgaagcagcagaatga

SEQ ID NO: 24 - DNA, 1503 bp, Rhodobacter capsulatus
atgaaaatctggctgccgtgcgatgctgctgctaaagcgtgtggtgcggaagcggttctggctgccctgcgtctggaagcggaaaaacgtggcggtgcactggatattgctcgtaacggcagccgcggtatgatctggctggaaccgctgctggaagtggaaaccccggccggtcgtattggttttggtccgatgacgccggccgatgttccggcactgtttgacgcactggaatctcatccgaaagccctgggcctggtcgaagaaattccgtttttcaaacgtcagacccgcctgacgtttgcccgttgtggtcgcatcgaaccgctgtcactggcacaatttgccgcggcagaaggttgggctggtctgcgtaaagcgctgaaaatgaccccggccgaagtggttgaagaagttctggcatcgggtctgcgcggtcgcggcggtgcaggttttccgaccggtattaaatggcgcaccgttgctgccgcacaggcggatcaaaaatacatcgtgtgtaatgttgatgaaggcgacagcggttcttttgcggatcgcatgctgattgaaggtgacccgttctgcctggttgaaggcatggcgattgcgggtcatgcagttggtgctacccgtggctatgtgtacattcgctcagaatacccggacgcaattgctgtgatgcgtgcagctatcgcgatggccaaaccgtttctggcagaagctggcttcgaaatggaagtgcgtgttggtgccggtgcatatgtgtgcggtgaagaaaccagtctgctgaactccctggaaggcaaacgtggtaccgttcgtgcaaaaccgccgctgccggcactgaaaggcctgtttggtaaaccgaccgtcgtgaacaatctgctgtccctggcggccgttccgtggattatcgcgcacggtgcaaaagcttatgaatcattcggtatggatcgtagccgtggtaccattccgctgcagatcggcggtaatgtgaaacgtggcggtctgtttgaaaccggcttcggtattacgctgggtgaactggttgaagatatttgcggcggtaccgctagtggtcgcccggtcaaagccgtccaagtgggcggtccgctgggtgcatatcatccggtctccgactaccacctgccgttttgctatgaacagtttgctggtcaaggcggtctggtgggccatgcaggtctggttgtccacgatgacaccgcggatatgctgaaactggcgcgttttgccatggaattctgtgcgattgaaagctgcggtacctgtacgccgtgccgtattggtgcggttcgcggcgttgaagtcattgatcgtattgcagctggtgatgcgagcgcaatgccgctgctggatgacctgtgtcagaccatgaaactgggttctctgtgtgccctgggcggttttacgccgtacccggtgcagtcggctatccgtcattttccggctgattttccgtgtgctcgtgaagcggcagaataa

SEQ ID NO: 25 - DNA, 453 bp, Rhodobacter capsulatus
atgacggataccgctcgtctgcgtgcgattctggccgcccaccgtggtcgtgaaggcgctctgctgccgattctgcatgatgtccaagcggcctttggcttcattccggaagatgcatatgctccgatcgcagctgacctgggtctgacccgtgcagaagtggctggcgtggttggtttttaccatgatttccgtaaagcgccggccggccgccacgttattaaactgtgccgcgcagaagcttgtcaggcaatgggtatggatgcggtccaagcgcgcctggaaagtgcactgggtctgcgtctgggtgacagctctgaagcagtcacgctggaagctgtgtattgtctgggtctgtgtgcctgtgcaccggccgcaatggttgatgaccgtctggttggtcgtctggatgcggcggcggtggcgggtatcgtggcggaactgggtgcgtga

SEQ ID NO: 26 - DNA, 768 bp, Rhodobacter capsulatus
atgtccctgccggctggtgctgttacggttccgctgccgggtggtgctcgtgctgttctggccgaagaagttccggtggcgctggtcttcgacggcgttacccaggcagtgatgatggcgagcccggtggatctggaagactttctgctgggttttgcactgaccgaaggcatgattgcggatcgtgctgaactgctgcgtcatgaagtggttcgccagccgcaaggtattgaactgcgcggttggctggctgcaccggccggccagcgttttgcggcacgccgtcgtgcgatggcaggcccggttggttgtggcctgtgtggtctggatagcctggcagcggttctgcgtccgctgccgcgtgcaccgcgcggcggtgcgccgccgccgctggcggatggtgctctggcggcactgcgcgcgggccaaagcctgcaggatgcagtgcgtagcgttcatgcagcgggcttttgggatggcgcgcagatgcgtgccctgcgtgaagatgttggccgtcataatgcgctggataaactggcgggcgctctggcaggccagggtatcgatgctgcggcaggtgcactggtgctgacctcacgtctgtccgttgatctggtgcaaaaagcagcgatgattggcgcgcgtgtgctgattgcgccgtctgcaccgaccgctctggcggtcgcagaagcgcaggcagccggcctggcactgattgcccgtggcccggatggtccgacgctgtacaccgaaaccgaagcagaatga

SEQ ID NO: 27 - DNA, 216 bp, Rhodobacter capsulatus
atgagtgacgacaaaattatccgtatggcaaatcaaatcgctgcgttcttcgccgttcaaccgggtgaccgtgcgggtccggtggcagcgcatattagcgaaaactggtctgcaccgatgcgtgccgcactgctggcgcacgtggcagctcagagtccgggtctggatccgctggttatcgcagcagcaccgcaaattcgtccggttccggcgtga

SEQ ID NO: 28 - DNA, 4414 bp, Artificial Sequence
  pTrcHis vector
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggggggttctcatcatcatcatcatcatggtatggctagcatgactggtggacagcaaatgggtcgggatctgtacgacgatgacgataaggatcgatggggatccgagctcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 29 - DNA, 10249 bp, Artificial Sequence
  plasmid comprising RcFDH and His-tag
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggggggttctcatcatcatcatcatcatggtatggctagcatgacggataccgctcgtctgcgtgcgattctggccgcccaccgtggtcgtgaaggcgctctgctgccgattctgcatgatgtccaagcggcctttggcttcattccggaagatgcatatgctccgatcgcagctgacctgggtctgacccgtgcagaagtggctggcgtggttggtttttaccatgatttccgtaaagcgccggccggccgccacgttattaaactgtgccgcgcagaagcttgtcaggcaatgggtatggatgcggtccaagcgcgcctggaaagtgcactgggtctgcgtctgggtgacagctctgaagcagtcacgctggaagctgtgtattgtctgggtctgtgtgcctgtgcaccggccgcaatggttgatgaccgtctggttggtcgtctggatgcggcggcggtggcgggtatcgtggcggaactgggtgcgtgaatattaaagaggtatatattaatgaaaatctggctgccgtgcgatgctgctgctaaagcgtgtggtgcggaagcggttctggctgccctgcgtctggaagcggaaaaacgtggcggtgcactggatattgctcgtaacggcagccgcggtatgatctggctggaaccgctgctggaagtggaaaccccggccggtcgtattggttttggtccgatgacgccggccgatgttccggcactgtttgacgcactggaatctcatccgaaagccctgggcctggtcgaagaaattccgtttttcaaacgtcagacccgcctgacgtttgcccgttgtggtcgcatcgaaccgctgtcactggcacaatttgccgcggcagaaggttgggctggtctgcgtaaagcgctgaaaatgaccccggccgaagtggttgaagaagttctggcatcgggtctgcgcggtcgcggcggtgcaggttttccgaccggtattaaatggcgcaccgttgctgccgcacaggcggatcaaaaatacatcgtgtgtaatgttgatgaaggcgacagcggttcttttgcggatcgcatgctgattgaaggtgacccgttctgcctggttgaaggcatggcgattgcgggtcatgcagttggtgctacccgtggctatgtgtacattcgctcagaatacccggacgcaattgctgtgatgcgtgcagctatcgcgatggccaaaccgtttctggcagaagctggcttcgaaatggaagtgcgtgttggtgccggtgcatatgtgtgcggtgaagaaaccagtctgctgaactccctggaaggcaaacgtggtaccgttcgtgcaaaaccgccgctgccggcactgaaaggcctgtttggtaaaccgaccgtcgtgaacaatctgctgtccctggcggccgttccgtggattatcgcgcacggtgcaaaagcttatgaatcattcggtatggatcgtagccgtggtaccattccgctgcagatcggcggtaatgtgaaacgtggcggtctgtttgaaaccggcttcggtattacgctgggtgaactggttgaagatatttgcggcggtaccgctagtggtcgcccggtcaaagccgtccaagtgggcggtccgctgggtgcatatcatccggtctccgactaccacctgccgttttgctatgaacagtttgctggtcaaggcggtctggtgggccatgcaggtctggttgtccacgatgacaccgcggatatgctgaaactggcgcgttttgccatggaattctgtgcgattgaaagctgcggtacctgtacgccgtgccgtattggtgcggttcgcggcgttgaagtcattgatcgtattgcagctggtgatgcgagcgcaatgccgctgctggatgacctgtgtcagaccatgaaactgggttctctgtgtgccctgggcggttttacgccgtacccggtgcagtcggctatccgtcattttccggctgattttccgtgtgctcgtgaagcggcagaataaatattaaagaggtatatattaatgaaagacctgattatcccgccgctggactggacgcaagacatgggtacgccgaaacgcgaaggtgctccggtgcatctgacgattgatggcgtcgaagtgacggttccggcgggtaccagcgttctgcgtgccgcagcagaagctggcatttctatcccgaaactgtgcgcgaccgataacgtggaaccggttggtagttgccgtctgtgtatggtggaaattgaaggcatgcgtggtaccccgacgtcctgtaccacgccggttgccccgggtatgcgtgtccatacccagacgccgcagctgcaaaaactgcgtcgcggtgtgatggaactgtatatcagcgatcacccgctggactgcctgacctgtgctgcgaacggcgattgcgaactgcaggacatggcaggcgctgtgggtctgcgtgaagttcgctatcaagcgaaagatacgcattttgcgcgtcgcgatgccaccggtccgaatccgcgttacattccgaaagataactcaaatccgtatttctcgtacgacccggccaaatgcatcgtctgtatgcgttgcgtgcgcgcctgcgaagaagtgcagggcacgtttgcactgaccgttatgggccgtggtttcgatgcgcgcattagtccggccgcaccggattttctgagcagcgactgtgtgtcctgcggcgcctgtgttcaggcatgtccgaccgctaccctggtggaaaaatctgtggaacgtatcggtaccccggaacgcaaagtggttaccacgtgcgcctattgtggcgttggttgctcgtttgaagcacatatgctgggcgatcagctggtccgtatggtgccgtggaaaggcggtgctgcgaaccgtggtcatagctgtgtgaaaggccgcttcgcgtatggttacgccacccaccaggatcgtattctgaaaccgatgattcgcgataaaatcacggacccgtggcgcgaagttaactggaccgaagcactggattttaccgctacgcgtctgcgcgcgctgcgtgatagccatggtgccgatgcactgggtgtgatcaccagttcccgctgcacgaatgaagaaacctatctggttcagaaactggcacgtgctgtcttcggtacgaacaataccgatacgtgcgcccgcgtttgtcattctccgaccggctacggtctgaaacagacctttggcacgagtgccggtacgcaagatttcgactccgtcgaagaaaccgatctggccctggtgattggcgcaaatccgaccgacggtcacccggtgtttgcgagtcgtctgcgtaaacgtctgcgtgccggtgcaaaactgattgtcgtggatccgcgtcgcatcgacctgctgaacaccccgcatcgtggcgaagcatggcacctgcagctgaaaccgggtacgaatgtcgcagtgatgaccgcaatggctcatgttattgtcaccgaacaaatctttgataaacgtttcatcggtgaccgctgcgattgggacgaatgggcggattatgccgaattcgtggcgaacccggaatacgcaccggaagctgtcgaaagcctgaccggcgtgccggctggtctgctgcgtcaggcggcacgtgcctatgctgccgcaccgaatgcagctatttattacggcctgggtgtgaccgaacacagccaaggctctaccacggttattgcgatcgccaacctggccatgatgaccggcaatatcggtcgtccgggcgtgggtgttaacccgctgcgtggtcagaacaatgtgcaaggcagctgtgatatgggttcttttccgcatgaattcccgggttatcgccacgttagtgatgacgcgacccgtggcctgtttgaacgcacgtggggtgtgaccctgtcatcggaaccgggcctgcgtattccgaatatgctggatgcggccgttgaaggccgcttcaaagcgctgtatgtccagggtgaagacattctgcaatcagatccggacacccgtcatgtttcggccggtctggcagctatggatctggtcatcgtgcatgacctgtttctgaacgaaacggcgaattacgcccacgtgtttctgccgggctccaccttcctggaaaaagatggtacctttacgaacgcggaacgtcgcattaatcgcgttcgtcgcgtcatggcaccgaaagctggtttcgcggattgggaagtgacgcagatgctggcaaacgctctgggcgctggttggcattatacccacccgtcagaaattatggcggaaattgccgcaaccacgccgggttttgcagctgtcacctacgaaatgctggatgcacgtggttcagtgcagtggccgtgcaatgaaaaagcgccggaaggctcgccgattatgcatgtcgaaggttttgtgcgcggcaaaggtcgtttcatccgcaccgcgtatctgccgacggatgaaaaaaccggtccgcgtttcccgctgctgctgaccacgggccgcattctgagccagtacaacgttggtgcccaaacccgtcgcacggaaaataccgtctggcatggtgaagatcgcctggaaattcacccgacggacgcagaaacccgtggcatccgcgatggtgattgggttcgtctggccagccgtgcaggtgaaaccaccctgcgtgcaaccgttacggatcgcgtctctccgggtgttgtctataccacgtttcatcacccggacacccaggcgaacgtggttaccacggatacgtctgactgggctaccaattgtccggaatacaaagtgaccgcggttcaagtcgcggccagtaatggcccgtccgattggcaacaggactatgcggcacaagcagcagcagcccgtcgtattgaagcagcagaatgaatattaaagaggtatatattaatgtccctgccggctggtgctgttacggttccgctgccgggtggtgctcgtgctgttctggccgaagaagttccggtggcgctggtcttcgacggcgttacccaggcagtgatgatggcgagcccggtggatctggaagactttctgctgggttttgcactgaccgaaggcatgattgcggatcgtgctgaactgctgcgtcatgaagtggttcgccagccgcaaggtattgaactgcgcggttggctggctgcaccggccggccagcgttttgcggcacgccgtcgtgcgatggcaggcccggttggttgtggcctgtgtggtctggatagcctggcagcggttctgcgtccgctgccgcgtgcaccgcgcggcggtgcgccgccgccgctggcggatggtgctctggcggcactgcgcgcgggccaaagcctgcaggatgcagtgcgtagcgttcatgcagcgggcttttgggatggcgcgcagatgcgtgccctgcgtgaagatgttggccgtcataatgcgctggataaactggcgggcgctctggcaggccagggtatcgatgctgcggcaggtgcactggtgctgacctcacgtctgtccgttgatctggtgcaaaaagcagcgatgattggcgcgcgtgtgctgattgcgccgtctgcaccgaccgctctggcggtcgcagaagcgcaggcagccggcctggcactgattgcccgtggcccggatggtccgacgctgtacaccgaaaccgaagcagaatgaatattaaagaggtatatattaatgagtgacgacaaaattatccgtatggcaaatcaaatcgctgcgttcttcgccgttcaaccgggtgaccgtgcgggtccggtggcagcgcatattagcgaaaactggtctgcaccgatgcgtgccgcactgctggcgcacgtggcagctcagagtccgggtctggatccgctggttatcgcagcagcaccgcaaattcgtccggttccggcgtgagagctcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

(19) United States
(12) Patent Application Publication
(10) Pub. No.: US 2024/0279691 A1
(43) Pub. Date: Aug. 22, 2024

(54) METHOD FOR PRODUCING FORMIC ACID USING CARBON MONOXIDE DEHYDROGENASE AND FORMATE DEHYDROGENASE

(71) Applicant: UNIST(ULSAN NATIONAL INSTITUTE OF SCIENCE AND TECHNOLOGY), Ulsan (KR)

(72) Inventors: Yong Hwan Kim, Ulsan (KR); Jin Hee Lee, Ulsan (KR); Ho Wan Hwang, Ulsan (KR); Byoung Wook Jeon, Ulsan (KR); Suk Min Kim, Ulsan (KR); Sung Heuck Kang, Ulsan (KR)

(73) Assignee: UNIST(ULSAN NATIONAL INSTITUTE OF SCIENCE AND TECHNOLOGY), Ulsan (KR)

(21) Appl. No.: 18/000,892

(22) PCT Filed: Feb. 17, 2021
(86) PCT No.: PCT/KR2021/002023

§ 371 Date: Dec. 6, 2022

(30) Foreign Application Priority Data
Jan. 6, 2021 (KR) 10-2021-0001403

(51) Int. Cl. C12P 7/40; B01D 53/84; C12N 9/02

(52) U.S. Cl. CPC C12P 7/40; B01D 53/84; C12N 9/0008; C12Y 102/01002; C12Y 102/07004; B01D 2251/95; B01D 2255/804; B01D 2257/502; B01D 2258/025

(57) ABSTRACT

Provided are a composition, a device, a filter, a method and the like, which convert toxic carbon monoxide and/or carbon dioxide in waste gas to formic acid without by-products at room temperature and at room pressure by using carbon monoxide dehydrogenase and formic acid dehydrogenase. The composition, the device, the filter, the method and the like enable the removal of carbon monoxide which is emitted in a great amount from industries such as petrochemical and steel industry and tobacco combustion, household cooking appliances, and various boiler combustion, through a cigarette filter, an air purifier, a household cooking appliance suction filter, a gas boiler, etc. Accordingly, the production method can be variously applied.

### TECHNICAL FIELD

The present disclosure relates to a method of producing formic acid by using carbon monoxide dehydrogenase and formate dehydrogenase.

### BACKGROUND ART

Waste gas emitted by industries such as steel and petrochemical industries contains a significant amount of toxic carbon monoxide (CO). The carbon monoxide isolated to a high degree of purity can be a starting raw material from which various compounds can be made. However, since waste gas contains various impurities, it is technically difficult to directly use carbon monoxide contained in industrial waste gas as a raw material for chemical reaction. For this reason, most industries use a method of recovering some thermal energy by combusting industrial waste gas containing carbon monoxide. However, since this method generates and emits a large amount of carbon dioxide through combustion of carbon monoxide, there is a need to prepare other alternatives in such a background that social interest in reducing greenhouse gas has recently increased and regulations on large amount of carbon dioxide emissions are being implemented. Accordingly, there is a growing demand to use the carbon monoxide contained in the waste gas as a valuable carbon resource rather than simply burning the same.

Meanwhile, there is a study to convert waste gas containing carbon monoxide through the water gas shift reaction, which has been used for a long time in the chemical industry, and the reaction scheme is as follows:

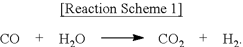

As suggested in Reaction Scheme 1 above, one molecule of hydrogen is produced by reacting carbon monoxide with water molecules. However, as can be seen from this reaction scheme, the same amount of carbon dioxide is generated as in the carbon monoxide combustion reaction, so that carbon monoxide cannot be used as a carbon resource. In addition, since it is relatively difficult to convert waste gas containing a large amount of impurities, it is necessary to purely separate carbon monoxide from waste gas in advance, which may increase the economic burden.

On the other hand, there have been quite a few attempts to convert carbon monoxide in waste gas into ethanol by using acetogen. The reaction scheme using the metabolic process of microorganisms is the same as Reaction Scheme 2:

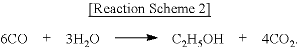

As can be seen from Reaction Scheme 2, some of the carbon of carbon monoxide is converted to carbon of ethanol to become a product, but most of carbon of carbon monoxide is not included in the product and is discharged as carbon dioxide. And this reaction is a reaction scheme in theory, and the growth and survival of microorganisms requires more energy, which is generated from the production of carbon dioxide. Accordingly, much more carbon dioxide emissions occur. In addition, although microorganisms have selectivity for carbon monoxide contained in waste gas, their survival rate can be easily affected by impurities, so it is difficult to stably produce ethanol.

Therefore, by actually realizing the reaction scheme that produces formic acid through the CO hydration reaction, which is a new reaction to remove carbon monoxide in waste gas, provided is a technology that uses carbon monoxide in waste gas as a carbon source and completely regenerates the same into formic acid with high added value without generating carbon dioxide.

### DESCRIPTION OF EMBODIMENTS

### Technical Problem

One aspect is to provide a composition for producing formic acid, the composition including carbon monoxide dehydrogenase and formate dehydrogenase.

One aspect is to provide a composition for removing at least one molecule selected from carbon monoxide and carbon dioxide, the composition including carbon monoxide dehydrogenase and formate dehydrogenase.

One aspect is to provide a composition for treating waste gas, the composition including carbon monoxide dehydrogenase and formate dehydrogenase.

One aspect is to provide a composition for air purification, the composition including carbon monoxide dehydrogenase and formate dehydrogenase.

One aspect is to provide a device for producing formic acid, the device including carbon monoxide dehydrogenase and formate dehydrogenase.

One aspect is to provide a device for removing at least one molecule selected from carbon monoxide and carbon dioxide, the device including carbon monoxide dehydrogenase and formate dehydrogenase.

One aspect is to provide a device for treating waste gas, the device including carbon monoxide dehydrogenase and formate dehydrogenase.

One aspect is to provide a device for air purification, the device including carbon monoxide dehydrogenase and formate dehydrogenase.

One aspect is to provide a filter including carbon monoxide dehydrogenase and formate dehydrogenase.

One aspect is to provide a method of producing a formic acid, the method including contacting a gas containing at least one molecule selected from carbon monoxide and carbon dioxide with carbon monoxide dehydrogenase and formate dehydrogenase.

One aspect is to provide a method of removing at least one molecule selected from carbon monoxide and carbon dioxide, the method including contacting a gas containing at least one molecule selected from carbon monoxide and carbon dioxide with carbon monoxide dehydrogenase and formate dehydrogenase.

One aspect is to provide a method of treating waste gas, the method including contacting a gas containing at least one molecule selected from carbon monoxide and carbon dioxide with carbon monoxide dehydrogenase and formate dehydrogenase.

One aspect is to provide a method of purifying air, the method including contacting a gas containing at least one molecule selected from carbon monoxide and carbon dioxide with carbon monoxide dehydrogenase and formate dehydrogenase.

### Solution to Problem

One aspect is to provide a composition for producing formic acid, the composition including carbon monoxide dehydrogenase and formate dehydrogenase.

The composition may enable the conversion into formic acid by bringing into contact with carbon monoxide and/or carbon dioxide. Specifically, the carbon monoxide dehydrogenase may convert carbon monoxide to carbon dioxide through Reaction Scheme 4:

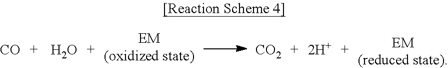

In addition, the formate dehydrogenase may convert carbon dioxide to formic acid through Reaction Scheme 5:

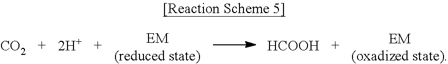

Therefore, the composition may convert carbon monoxide, in carbon monoxide or a gas containing carbon dioxide, into carbon dioxide, and the carbon dioxide into formic acid.

Therefore, the composition may convert carbon dioxide, in carbon dioxide or a gas containing carbon dioxide, into formic acid.

In addition, the composition can be reused while the production efficiency of formic acid does not fall.

The carbon monoxide dehydrogenase may be naturally derived, or may be obtained by various protein synthesis methods well known in the art. In an embodiment, carbon monoxide dehydrogenase may be prepared using polynucleotide recombination and a protein expression system, or synthesized in vitro through chemical synthesis such as protein synthesis, or prepared by a cell-free protein synthesis method. Also, in an embodiment, the carbon monoxide dehydrogenase may be a peptide, an extract of a plant-derived tissue or cell, or a product obtained by culturing a microorganism (for example, bacteria or fungi, and particularly yeast).

The term “protein” refers to a polymer consisting of two or more amino acids linked by amide bonds (or peptide bonds).

The term ‘expression’ refers to the process by which a polypeptide is produced from a structural gene. The process involves the transcription of a gene (polynucleotide) into mRNA and the translation of such mRNA into polypeptide (protein)(s).

The term ‘recombinant’ refers to a case where the cell replicates a heterologous nucleic acid, or expresses the nucleic acid, or expresses a peptide, a heterologous peptide, or a protein encoded by the heterologous nucleic acid. A recombinant cell may express genes or gene segments not found in the native form of the cell, either in a sense or antisense form. In addition, a recombinant cell may express a gene found in a cell in a natural state, but the gene is a modified gene, and re-introduced into the cell by artificial means.

The carbon monoxide dehydrogenase may include a protein having a sequence homology of about 70% or more, about 75% or more, about 80% or more, about 85% or more, about 90% or more, about 92% or more, about 95% or more, about 97% or more, about 98% or more, or about 99% or more, with respect to the amino acid sequence of the carbon monoxide dehydrogenase. The carbon monoxide dehydrogenase may be an isoenzyme of a carbon monoxide dehydrogenase, and, for example, may be derived from a microorganism belonging to at least one selected from Moorella sp., Rhodospirillum sp., Methanococcus sp., Methanosarcina sp., Methanothermobacter sp., Clostridium sp., Oligotropha sp., Aeropyrum sp., Ferroglobus sp. and Thermococcus sp.

In an embodiment, the carbon monoxide dehydrogenase may be a carbon monoxide dehydrogenase derived from at least one microorganism selected from Moorella thermoacetica, Rhodospirillum rubrum, Carboxydothermus hydrogenoformans, Methanococcus vannielii, Methanosarcina barkeri, Methanothermobacter thermautotrophicus, Clostridium pasteurianum, Oligotropha carboxidovorans, Aeropyrum pernix, Ferroglobus placidus, Clostridium autoethanogenum, Clostridium ragsdalei, Clostridium ljungdahlii, Clostridium scatologenes, Clostridium acetobutylicum, Clostridium beijerinckii, Clostridium perfringens, Clostridium thermocellum, Clostridium kluyveri, Clostridium botulinum, and Thermococcus onnurineus.

In an embodiment, the carbon monoxide dehydrogenase may be at least one carbon monoxide dehydrogenase derived from a Carboxydothermus hydrogenoformans-derived carbon monoxide dehydrogenase II, and a Carboxydothermus hydrogenoformans-derived carbon monoxide dehydrogenase IV, and Thermococcus onnurineus-derived carbon monoxide dehydrogenase.

Carboxydothermus hydrogenoformans-derived carbon monoxide dehydrogenase II may be a polypeptide consisting of the amino acid sequence of SEQ ID NO: 1, Carboxydothermus hydrogenoformans-derived carbon monoxide dehydrogenase IV may be a polypeptide consisting of the amino acid sequence of SEQ ID NO: 2, and Thermococcus onnurineus-derived carbon monoxide dehydrogenase may be a polypeptide consisting of the amino acid sequence of SEQ ID NO: 3.

In an embodiment, Carboxydothermus hydrogenoformans-derived carbon monoxide dehydrogenase II may be a polypeptide encoded by a polynucleotide consisting of the nucleotide sequence of SEQ ID NO: 11, Carboxydothermus hydrogenoformans-derived carbon monoxide dehydrogenase IV may be a polypeptide encoded by a polynucleotide consisting of the nucleotide sequence of SEQ ID NO: 12, and Thermococcus onnurineus-derived carbon monoxide dehydrogenase may be a polypeptide encoded by a polynucleotide consisting of the nucleotide sequence of SEQ ID NO: 13.

The term ‘homology’ is intended to indicate a degree of similarity with a wild-type amino acid sequence, and the comparison of such homology can be performed using a comparison program well known in the art, and the homology between two or more sequences can be calculated as a percentage (%).

In addition, to obtain better chemical stability, enhanced pharmacological properties (half-life, absorption, potency, efficacy, etc.), altered specificity (for example, broad spectrum of biological activity), and reduced antigenicity, a protecting group may be bonded to the N-terminus or C-terminus of the carbon monoxide dehydrogenase. The protecting group may be an acetyl group, a fluorenyl methoxycarbonyl group, a formyl group, a palmitoyl group, a myristyl group, a stearyl group, or a polyethylene glycol (PEG), and any component may be used as the protecting group as long as the component enhances the modification of the carbon monoxide dehydrogenase, particularly the stability of the carbon monoxide dehydrogenase.

The term “stability” may refer to not only in vivo stability that protects the carbon monoxide dehydrogenase from the attack by in vivo protein cleaving enzymes, but also storage stability (for example, room-temperature storage stability).

In addition, the carbon monoxide dehydrogenase may additionally include an amino acid sequence prepared for a specific purpose for a targeting sequence, a tag, and a labeled residue, and specifically may be in the form of binding to a His-tag terminal protein.

In an aspect, the formate dehydrogenase may be naturally derived, or may be obtained by various protein synthesis methods well known in the art. In an embodiment, the formate dehydrogenase may be prepared using polynucleotide recombination and a protein expression system, or synthesized in vitro through chemical synthesis such as protein synthesis, or prepared by a cell-free protein synthesis method. Also, in an embodiment, the formate dehydrogenase may be a peptide, an extract of a plant-derived tissue or cell, or a product obtained by culturing a microorganism (for example, bacteria or fungi, and particularly yeast).

The formate dehydrogenase may include a protein having a sequence homology of about 70% or more, about 75% or more, about 80% or more, about 85% or more, about 90% or more, about 92% or more, about 95% or more, about 97% or more, about 98% or more, or about 99% or more, with respect to the amino acid sequence of the formate dehydrogenase. The formate dehydrogenase may be an isoenzyme of formate dehydrogenase, and may be, for example, derived from a microorganism belonging to at least one of Methylobacterium sp., Thiobacillus sp., and Rhodobacter sp.

Specifically, the formate dehydrogenase may be derived from at least one microorganism selected from Methylobacterium extorquens, Thiobacillus sp. KNK65MA, and Rhodobacter capsulatus.

In an embodiment, the formate dehydrogenase may be at least one formate dehydrogenase selected from Methylobacterium extorquens-derived formate dehydrogenase I, Thiobacillus sp. KNK65MA-derived formate dehydrogenase, and Rhodobacter capsulatus-derived formate dehydrogenase.

The Methylobacterium extorquens-derived formate dehydrogenase I may consist of MeFDH I α subunit, which is a polypeptide having the amino acid sequence of SEQ ID NO: 4, and MeFDH I β subunit, which is a polypeptide consisting of the amino acid sequence of SEQ ID NO: 5; the Thiobacillus sp. KNK65MA-derived formate dehydrogenase may consist of a polypeptide consisting of the amino acid sequence of SEQ ID NO: 6; and the Rhodobacter capsulatus-derived formate dehydrogenase may consist of RcFDH a subunit, which is a polypeptide consisting of the amino acid sequence of SEQ ID NO: 7, RcFDH β subunit, which is a polypeptide consisting of the amino acid sequence of SEQ ID NO: 8, and a RcFDH γ subunit, which is a polypeptide consisting of the amino acid sequence of SEQ ID NO: 9.

In an embodiment, the Methylobacterium extorquens-derived formate dehydrogenase I may be a polypeptide encoded by fdh1A, which is a polynucleotide consisting of the nucleotide sequence of SEQ ID NO: 17 and qfdh1B, which is a polynucleotide consisting of the nucleotide sequence of SEQ ID NO: 18, the Thiobacillus sp. KNK65MA-derived formate dehydrogenase may be a polypeptide encoded by a polynucleotide consisting of the nucleotide sequence of SEQ ID NO: 21, and the Rhodobacter capsulatus-derived formate dehydrogenase may be a polypeptide encoded by fdsA, which is a polynucleotide consisting of the nucleotide sequence of SEQ ID NO: 23, fdsB, which is a polynucleotide consisting of the nucleotide sequence of SEQ ID NO: 24, fdsG, which is a polynucleotide consisting of the nucleotide sequence of SEQ ID NO: 25, fdsC, which is a polynucleotide consisting of the nucleotide sequence of SEQ ID NO: 26, and fdsD, which is a polynucleotide consisting of the nucleotide sequence of SEQ ID NO: 27.

In addition, to obtain better chemical stability, enhanced pharmacological properties (half-life, absorption, potency, efficacy, etc.), altered specificity (for example, broad spectrum of biological activity), and reduced antigenicity, a protecting group may be bonded to the N-terminus or C-terminus of the formate dehydrogenase. The protecting group may be an acetyl group, a fluorenyl methoxycarbonyl group, a formyl group, a palmitoyl group, a myristyl group, a stearyl group, or a polyethylene glycol (PEG), and any component may be used as the protecting group as long as the component enhances the modification of the formate dehydrogenase, particularly the stability of the formate dehydrogenase.

In addition, the formate dehydrogenase may additionally include an amino acid sequence prepared for a specific purpose for a targeting sequence, a tag, and a labeled residue, and specifically may be in the form of binding to a His-tag terminal protein.

Also, in one aspect, the composition for preparing formic acid may further include an electron mediator.

When the composition further includes an electron mediator, formic acid production efficiency can be increased.

The electron mediator may be a natural electron mediator or an artificial electron mediator. In an embodiment, the electron mediator may be at least one electron mediator selected from an electron mediator having a viologen group and an electron mediator having an adenine dinucleotide group, and may be, for example, at least one electron mediator selected from alkyl viologen, benzyl viologen, nicotinamide adenine dinucleotide (NAD), and flavin adenine dinucleotide (FAD), and the alkyl viologen may include methyl viologen, ethyl viologen, propyl viologen, and the like.

In an embodiment, the electron mediator may be at least one electron mediator selected from methyl viologen, ethyl viologen, benzyl viologen, nicotinamide adenine dinucleotide (NAD), and flavin adenine dinucleotide (FAD), and, for example, at least one electron mediator selected from methyl viologen and ethyl viologen.

Also, in one aspect, the carbon monoxide dehydrogenase and the formate dehydrogenase in the composition may be present in a ratio of 1:1 to 1:3.

In an embodiment, the carbon monoxide dehydrogenase and the formate dehydrogenase in the composition may be present in a ratio of 1:1 to 1:3, 1:1 to 1:2.5, 1:1 to 1:2.25, 1.5:1 to 1.5:3, 1.5:1 to 1.5:2.5, 1.5:1 to 1.5:2.25, 1.75:1 to 1.75:3, 1.75:1 to 1.75:2.5, or 1.75:1 to 1.75:2.25, wherein the ratio may be a molar ratio.

When the ratio of the carbon monoxide dehydrogenase and the formate dehydrogenase in the composition is 1:1 to 1:3, formic acid can be maximally produced.

Also, in one aspect, the pH of the composition may be from 5.0 to 8.0, from 5.0 to 7.5, from 5.0 to 7.0, from 5.5 to 8.0, from 5.5 to 7.5, from 5.5 to 7.0, from 6.0 to 8.0, from 6.0 to 7.5, or from 6.0 to 7.0.

When the pH of the composition is less than 5.0, the activity of the carbon monoxide dehydrogenase may be reduced, and when the pH of the composition is greater than 8.0, the activity of the formate dehydrogenase is reduced, and the formic acid production efficiency may be reduced.

One aspect is to provide a composition for removing at least one molecule selected from carbon monoxide and carbon dioxide, the composition including carbon monoxide dehydrogenase and formate dehydrogenase.

The ‘carbon monoxide dehydrogenase’, and the ‘formate dehydrogenase’ may be within the scopes described above.

The composition may remove carbon monoxide and/or carbon dioxide by conversion into formic acid through the contact with carbon monoxide and/or carbon dioxide, and may be reused without deterioration in the removal efficiency. A specific removal mechanism for carbon monoxide and/or carbon dioxide may be within the scopes described above.

One aspect is to provide a composition for treating waste gas, the composition including carbon monoxide dehydrogenase and formate dehydrogenase.

The ‘carbon monoxide dehydrogenase’, and the ‘formate dehydrogenase’ may be within the scopes described above.

The waste gas may be a large amount of waste gas discharged during combustion of industries such as petrochemicals and steel industry, tobacco combustion, household cooking appliances, and various boilers, and specifically may be a waste gas containing a large amount of carbon monoxide and/or carbon dioxide.

The composition may treat waste gas by removing carbon monoxide and/or carbon dioxide through conversion into formic acid via the contact with carbon monoxide and/or carbon dioxide in waste gas, and may be reused without deterioration in the treatment efficiency. A specific removal mechanism for carbon monoxide and/or carbon dioxide may be within the scopes described above.

One aspect is to provide a composition for air purification, the composition including carbon monoxide dehydrogenase and formate dehydrogenase.

The ‘carbon monoxide dehydrogenase’, and the ‘formate dehydrogenase’ may be within the scopes described above.

The composition may purify air by removing carbon monoxide and/or carbon dioxide through conversion into formic acid via the contact with carbon monoxide and/or carbon dioxide in air and may be reused without deterioration in the air purification efficiency. A specific removal mechanism for carbon monoxide and/or carbon dioxide may be within the scopes described above.

One aspect is to provide a device for producing formic acid, the device including carbon monoxide dehydrogenase and formate dehydrogenase.

The ‘carbon monoxide dehydrogenase’, the ‘formate dehydrogenase’, and the ‘formic acid’ may be within the scopes described above.

The device may contact carbon monoxide and/or carbon dioxide to convert into formic acid, and can be reused without lowering manufacturing efficiency. In an embodiment, the device may convert carbon monoxide into carbon dioxide, in carbon monoxide or a gas containing carbon monoxide, and carbon dioxide, and the carbon dioxide into formic acid.

Therefore, the device may be used to prepare formic acid by converting carbon dioxide, in carbon dioxide or a gas containing carbon dioxide, into formic acid. Specific formic acid production mechanism may be within the scopes described above.

One aspect is to provide a device for removing at least one molecule selected from carbon monoxide and carbon dioxide, the device including carbon monoxide dehydrogenase and formate dehydrogenase.

The ‘carbon monoxide dehydrogenase’, and the ‘formate dehydrogenase’ may be within the above-described range.

The device may remove carbon monoxide and/or carbon dioxide by conversion into formic acid through the contact with carbon monoxide and/or carbon dioxide, and may be reused without deterioration in the removal efficiency. A specific removal mechanism for carbon monoxide and/or carbon dioxide may be within the scopes described above.

One aspect is to provide a device for treating waste gas, the device including carbon monoxide dehydrogenase and formate dehydrogenase.

The ‘carbon monoxide dehydrogenase’, the ‘formate dehydrogenase’, and the ‘waste gas’ may be within the scopes described above.

The device may treat waste gas by removing carbon monoxide and/or carbon dioxide through conversion into formic acid via the contact with carbon monoxide and/or carbon dioxide in waste gas, and may be reused without deterioration in the treatment efficiency. A specific removal mechanism for carbon monoxide and/or carbon dioxide may be within the range described above.

One aspect is to provide a device for air purification, the device including carbon monoxide dehydrogenase and formate dehydrogenase.

The ‘carbon monoxide dehydrogenase’, and the ‘formate dehydrogenase’ may be within the above-described range.

The device may purify air by removing carbon monoxide and/or carbon dioxide through conversion into formic acid via the contact with carbon monoxide and/or carbon dioxide in air and may be reused without deterioration in the air purification efficiency. A specific removal mechanism for carbon monoxide and/or carbon dioxide may be within the range described above.

One aspect is to provide a filter including carbon monoxide dehydrogenase and formate dehydrogenase.

The ‘carbon monoxide dehydrogenase’, and the ‘formate dehydrogenase’ may be within the above-described range.

The filter may remove carbon monoxide and/or carbon dioxide by conversion into formic acid through the contact with carbon monoxide and/or carbon dioxide, and may be reused without deterioration in the removal efficiency. A specific removal mechanism for carbon monoxide and/or carbon dioxide may be within the range described above.

The filter can be applied to various filters, such as cigarette filters and air purifier filters, in places where carbon monoxide and/or carbon dioxide is generated, and may be used for industrial sites requiring hazardous gas treatment technology, sterilization/removal purification technologies and system for harmful substances in air, treatment facilities for indoor air quality management in vehicles, trains, etc. and technologies related theretotechnologies and devices for ventilation efficiency and economical ventilation, an indoor air purification device such as an air purifier, an air conditioner, or a ventilator.

One aspect is to provide a method of producing a formic acid, the method including contacting a gas containing at least one molecule selected from carbon monoxide and carbon dioxide with carbon monoxide dehydrogenase and formate dehydrogenase.

The ‘carbon monoxide dehydrogenase’, the ‘formate dehydrogenase’, and the ‘formic acid’ may be within the scopes described above.

By contacting carbon monoxide and/or carbon dioxide with carbon monoxide dehydrogenase and formate dehydrogenase, formic acid can be easily and efficiently produced at room temperature and room pressure.

In an embodiment, the carbon monoxide dehydrogenase converts carbon monoxide, in carbon monoxide and/or a gas containing carbon monoxide, into carbon dioxide, and continuously, the formate dehydrogenase converts carbon dioxide into formic acid to produce formic acid.

In an embodiment, the formate dehydrogenase may convert carbon dioxide, in carbon dioxide or a gas containing carbon dioxide, into formic acid, thereby producing formic acid. Specific formic acid production mechanism may be within the scopes described above.

In addition, carbon monoxide dehydrogenase and/or formate dehydrogenase can be reused while formic acid production efficiency thereof is not reduced.

Also, in one aspect, the contacting may include: contacting the gas with carbon monoxide dehydrogenase; and contacting, with the formate dehydrogenase, the gas which has been in contact with the carbon monoxide dehydrogenase.

In the method of producing the formic acid, the gas may be simultaneously brought into contact with carbon monoxide dehydrogenase and formate dehydrogenase. However, in other embodiments, the contacting may be performed sequentially: carbon monoxide dehydrogenase and then formate dehydrogenase.

Also, in one aspect, the gas may be a continuously supplied gas.

In the case where the gas is continuously supplied, since carbon monoxide dehydrogenase and/or formate dehydrogenase can be reused, formic acid may be continuously generated.

When the amount of the continuously supplied gas is increased, the formic acid production rate may also be increased.

In addition, in one aspect, the gas may be in contact with carbon monoxide dehydrogenase and formate dehydrogenase, simultaneously with the electron mediator.

The “electron mediator” may be within the scope described above, and when the electron mediator is additionally in contact, the formic acid production efficiency may be increased.

One aspect is to provide a method of removing at least one molecule selected from carbon monoxide and carbon dioxide, the method including contacting a gas containing at least one molecule selected from carbon monoxide and carbon dioxide with carbon monoxide dehydrogenase and formate dehydrogenase.

The ‘gas’, ‘carbon monoxide dehydrogenase’, and the ‘formate dehydrogenase’ may be within the scopes described above.

By bringing the gas into contact with the carbon monoxide dehydrogenase and the formate dehydrogenase, carbon monoxide and/or carbon dioxide can be removed, and the enzymes can be reused without deterioration in removal efficiency. A specific removal mechanism for carbon monoxide and/or carbon dioxide may be within the scopes described above.

One aspect is to provide a method of treating waste gas, the method including contacting a gas containing at least one molecule selected from carbon monoxide and carbon dioxide with carbon monoxide dehydrogenase and formate dehydrogenase.

The ‘gas’, ‘carbon monoxide dehydrogenase’, the ‘formate dehydrogenase’, and the ‘waste gas’ may be within the scopes described above.

By bringing the gas into contact with the carbon monoxide dehydrogenase and the formate dehydrogenase, waste gas may be treated by removing carbon monoxide and/or carbon dioxide, and the enzymes can be reused without deterioration in removal efficiency. A specific removal mechanism for carbon monoxide and/or carbon dioxide may be within the range described above.

One aspect is to provide a method of purifying air, the method including contacting a gas containing at least one molecule selected from carbon monoxide and carbon dioxide with carbon monoxide dehydrogenase and formate dehydrogenase.

The ‘gas’, ‘carbon monoxide dehydrogenase’, and the ‘formate dehydrogenase’ may be within the scopes described above.

By bringing the gas into contact with the carbon monoxide dehydrogenase and the formate dehydrogenase, air may be purified by removing carbon monoxide and/or carbon dioxide, and the enzymes can be reused without deterioration in removal efficiency. A specific removal mechanism for carbon monoxide and/or carbon dioxide may be within the range described above.

### Advantageous Effects of Disclosure

According to a method of producing formic acid according to an aspect, toxic carbon monoxide and/or carbon dioxide in waste gas can be converted into formic acid without by-products at room temperature and at room pressure by using carbon monoxide dehydrogenase and formate dehydrogenase, and the enzymes can be reused without any decrease in efficiency thereof. The method of producing formic acid according to an aspect enables the removal of carbon monoxide which is emitted in a great amount from industries such as petrochemical and steel industry and tobacco combustion, household cooking appliances, various boiler combustion, through a cigarette filter, an air purifier, a household cooking appliance suction filter, a gas boiler, etc. Accordingly, the production method can be variously applied.

### BRIEF DESCRIPTION OF DRAWINGS

FIG. 1 shows a conceptual diagram of enzymes catalyzing the CO hydration reaction and production of formic acid (EV is ethyl viologen, a compound used as an electron mediator).

FIG. 2 show a diagram schematically illustrating free energy values according to an electron mediator (Mdₒₓ: an oxidized electron mediator, assuming that the free energy in the initial state of the reaction is 0).

FIG. 3 shows a diagram illustrating relative activity changes of ChCODH II (black) and MeFDH I (red) according to the reaction pH.

FIG. 4 shows a diagram of the time-dependent change of formic acid concentration of the CO hydration enzyme reaction tested by continuously introducing gas into a 100 mL bubble column reactor (50% CO and 50% CO₂ gas are used).

FIG. 5 shows a diagram of the time-dependent formic acid concentration when the CO hydration reaction is performed using a crude gas (black) containing 50% of CO and an actual waste gas (red) emitted from the steel industry.

FIG. 6 shows a diagram confirming the possibility of repeated use of the CO hydration enzyme reaction (black: 50% CO, 50% CO₂ gas; red: waste gas LDG generated by the steel industry).

### MODE OF DISCLOSURE

Hereinafter, the present disclosure will be described in more detail through examples. However, these examples are for illustrative purposes of the present disclosure, and the scope of the present disclosure is not limited to these examples.

### REFERENCE EXAMPLE

Examples present a novel carbon monoxide hydration reaction as shown in Reaction Scheme 3:

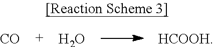

Referring to Reaction Scheme 3, it can be seen that formic acid is produced by bonding a water molecule to a carbon monoxide molecule, and the carbon atom originally contained in carbon monoxide is completely converted into the carbon atom of formic acid without formation of any carbon dioxide. In order to proceed with this reaction, two enzymes are used together as shown in Reaction Schemes 4 and 5 presented below:

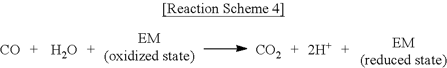

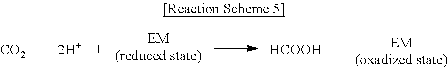

EM: electron mediator.

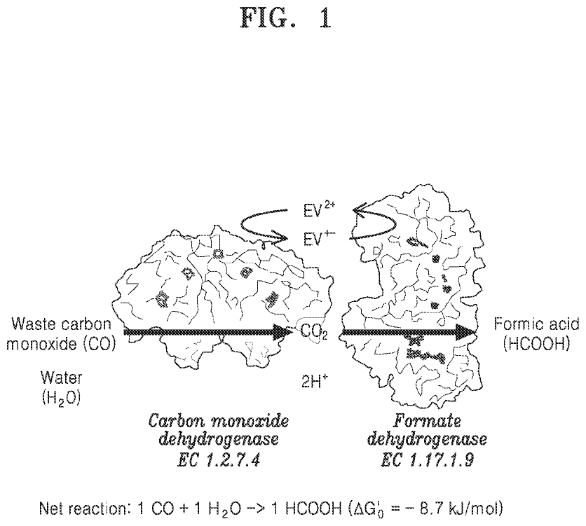

The reaction of Reaction Scheme 4 is catalyzed by CO dehydrogenase, an enzyme that oxidizes CO, and the reaction of Reaction Scheme 5 is catalyzed by formate dehydrogenase. A conceptual diagram of these is shown in FIG. 1.

As can be seen in FIG. 1, the difference in free energy of formic acid production in the reaction of Reaction Scheme 3 is −8.7 KJ/mol, which is a negative value, indicating that the reaction can proceed without input of external energy. In order to perform a smooth reaction, in addition to the enzymes (CO dehydrogenase, formate dehydrogenase) that catalyze the reaction, an electron mediator needs to be able to transfer electrons between these two enzymes. In order to transfer electrons, the electron mediator needs to be able to react with the enzymes, and at the same time, retains the reducing power generated by oxidizing CO to such a state that is sufficiently to reduce CO₂.

### EXAMPLE

### Example 1. Preparation of Carbon Monoxide Dehydrogenase (CODH) and Formate Dehydrogenase (FDH)

### (1) Cloning of CODH and FDH

The expression and purification process of ChCODH II (SEQ ID NO: 1), ChCODH IV(SEQ ID NO: 2), ToCODH(SEQ ID NO: 3)(respectively corresponding to Carboxydothermus-derived II, IV types of CODH and Thermococcus onnurineus-derived CODH), MeFDH I (MeFDH I a subunit: SEQ ID NO: 4; MeFDH I β subunit: SEQ ID NO: 5), TsFDH(SEQ ID NO: 6), and RcFDH(RcFDH α subunit: SEQ ID NO: 7; RcFDH β subunit: SEQ ID NO: 8; RcFDH γ subunit: SEQ ID NO: 9)(respectively corresponding to Methylobacterium extorquens-derived I type of FDH, Thiobacillus sp. KNK65MA-derived FDH and Rhodobacter capsulatus-derived FDH) were as follows.

In the case of CODHs (ChCODH II, ChCODH IV and ToCODH), first, the CODH genes (ChCODH II gene: SEQ ID NO: 11; ChCODH IV gene: SEQ ID NO: 12; ToCODH gene: SEQ ID NO: 13), which were to be tested, were each cloned in pET-28a(+) vector (SEQ ID NO: 10) together with a vector containing a His-tag. These plasmids (ChCODH II+His-tag containing plasmid: SEQ ID NO: 14; ChCODH IV+His-tag containing plasmid: SEQ ID NO: 15; ToCODH+His-tag containing plasmid: SEQ ID NO: 16) were each, together with pRKISC, placed in Escherichia coli BL21(DE3) and transformed and then expressed.

In the case of MeFDH I, the FDH gene (fdh1A: SEQ ID NO: 17; fdh1B: SEQ ID NO: 18) was cloned into pCM110 vector (SEQ ID NO: 19) together with a His-tag. And this plasmid (a plasmid including MeFDH I gene and His-tag: SEQ ID NO: 20) was added to Methylobacterium extorquens AM1 in which the FDH gene was deleted, and transformed.

As for TsFDH, a plasmid (a plasmid containing TsFDH gene and His-tag: SEQ ID NO: 22) created by cloning the FDH gene (SEQ ID NO: 21) into pET-23b(+) vector with His-tag, was transformed in E. coli BL21(DE3), and then expressed.

As for RcFDH, a plasmid (a plasmid containing RcFDH gene and His-tag: SEQ ID NO: 29) created by cloning FDH gene (fdsA: SEQ ID NO: 23; fdsB: SEQ ID NO: 24; fdsG: SEQ ID NO: 25; fdsC: SEQ ID NO: 26; and fdsD: SEQ ID NO: 27) into pTrcHis vector (SEQ ID NO: 28), together with His-tag, was transformed into E. coli MC1061 and then expressed.

### (2) Expression of CODH

ChCODH II, ChCODH IV, and ToCODH were all expressed in the same manner. First, the strain prepared in LB medium containing 50 μg/mL of kanamycin and 10 μg/mL of tetracycline was cultured in a shaking incubator at 37° C. and 200 rpm for 16 hours. Thereafter, 5 mL of the cultured strain was added to 400 mL of TB medium containing 0.02 mM NiCl₂, 0.1 mM FeSO₄, and 2 mM and the same concentration of antibiotic as before (12 g/L trypton, 24 g/L Yeast extract, 10 g/L NaCl, 11 g/L glycerol, 12.3 g/L K₂HPO₄, and 2.2 g/L KH₂PO₄). After that, culturing was performed in a 1 L Erlenmeyer flask at 37° C. and 200 rpm until an OD₆₀₀ of 0.4 to 0.6 was reached. The cultured bacteria were transferred to a 500 mL serum bottle and purged with nitrogen for 1 hour to exchange dissolved gas. When 30 minutes had elapsed during the gas exchange process, 0.5 mM NiCl₂, 1 mM FeSO₄, 50 mM KNO₃, and 0.2 mM IPTG (Isopropyl β-D-1-thiogalactopyranoside) were added thereto express the protein. After gas exchange, the cells were cultured at 30° C. and 200 rpm for 24 hours, and the cultured cells were harvested and stored. The amount to be used for driving the 10 L reactor was cultured through a 100 L incubator with reference to this method.

### (3) Expression of FDH

### 1) Expression of MeFDH I

Prepared was a medium containing 1.62 g/L NH₄Cl, 0.2 g/L MgSO₄, 2.21 g/L K₂HPO₄, 1.25 g/L NaH₂PO₄2H₂O, 15 mg/L Na₂EDTA₂H₂O, 4.5 mg/L ZnSO+7H₂O, 0.3 mg/L CoCl₂6H₂O, 1 mg/L MnCl₂4H₂O, 1 mg/L HsBO₃, 2.5 mg/L CaCl₂), 0.4 mg/L Na₂MoO₄2H₂O, 3 mg/L FeSO₄7H₂O, 0.3 mg/L CuSO+5H₂O, 30 μM Na₂WO₄, 16 g/L succinate, 50 μg/mL rifamycin, and 10 μg/mL tetracycline. The strain prepared in the medium was cultured in a shaking incubator at 30° C. and 200 rpm for 72 hours. 200 ml of the same medium except for Rifamycin was placed in a 1 L Erlenmeyer flask, 2 mL of the cultured strain was added thereto, and cultured under the same conditions until OD₆₀₀ of 0.4˜ 0.6 was reached. Protein expression was induced by adding 0.5 wt % of methanol, then cultured for 24 hours, harvested and stored. The amount to be used for driving the 10 L reactor was cultured through a 100 L incubator with reference to this method.

### 2) Expression of TsFDH

Pre-culture was cultured in 3 mL of LB medium containing 50 μg/mL of ampicillin in a shaking incubator at 37° C. and 200 rpm. The pre-cultured cells were placed in 300 mL of medium supplemented with 1 mM IPTG and expressed in a shaking incubator at 37° C. and 200 rpm for 24 hours.

### 3) Expression of RcFDH

Preculturing was performed in a LB medium containing the additives of 1 mM molybdate, 20 μM IPTG, and 150 μg/mL ampicillin for 12 hours at 37° C. The strain which was pre-cultured at the ratio of 1:500, was placed in a medium of the same composition, and cultured. During the culture, the cells were cultured for 24 hours in a shaking incubator at 30° C. and 130 rpm.

### (4) Purification of CODH

50 mM KH₂PO₄, 300 mM NaCl, 10 mM imidazole, 2 mM dithioerythritol (DTE), 2 μM resazurin, lysis buffer having the pH of 8.0, 50 mM NaH₂PO₄, 300 mM NaCl, 20 mM imidazole, 2 mM DTE, 2 UM resazurin, wash buffer having the pH of 8.0, 50 mM NaH₂PO₄, 300 mM NaCl, 250 mM imidazole, 2 mM DTE, 2 UM resazurin, and elution buffer having the pH of 8.0 were prepared. 10 mL of lysis buffer per 1 g of harvested cell pellet in an anaerobic chamber was mixed therewith and released with a pipette. Cells were lysed by ultrasound for 30 minutes per 1 g of cell pellet, and then subjected to centrifuging at 11000 rpm and 4ºC for 20 minutes and only supernatant was taken therefrom. 1 mL of Ni-NTA agarose based on 1 g of cell pellet, and supernatant were mixed and pipetted to bind the expressed protein for 15 minutes. The binding solution was poured onto the column, and 10 cv of wash buffer was poured and washed. In the experiment of continuous gas inflow, only a part thereof was eluted with elution buffer to measure concentration and activity, and the remaining enzymes were used while being bound. In other experiments, elution was performed with an elution buffer, and when operating a 10 L reactor, cells were lysed using a homogenizer.

### (5) Purification of FDH

### 1) Purification of MeFDH I

50 mM MOPS, 300 mM NaCl, 20 mM imidazole, buffer A having the pH of 7.0, 50 mM MOPS, 300 mM NaCl, 300 mM imidazole, buffer B having the pH of 7.0 were prepared. 20 mL of buffer A per 1 g of the cell pellet harvested in an anaerobic chamber was mixed and released with a pipette. Then, while checking the OD₆₀₀, the cells were lysed by ultrasonication until the value became 30% or less of the initial value, and were centrifuged at 11000 rpm and at a temperature of 4ºC for 20 minutes and only the supernatant was taken therefrom. 1 mL of Ni-NTA agarose based on 1 g of cell pellet, and supernatant were mixed and pipetted to bind the expressed protein for 15 minutes. The binding solution was poured onto the column, and 10 cv of buffer A was poured and washed. In the experiment of continuous gas inflow, only a part thereof was eluted with buffer B to measure concentration and activity, and the remaining enzymes were used while being bound. In other experiments, elution was performed with buffer B, and when operating a 10 L reactor, cells were lysed using a homogenizer.

### 2) Purification of TsFDH

50 mM NaH₂PO₄, 300 mM NaCl, lysis buffer having the pH of 7.0, 40 mM imidazole, 50 mM NaH₂PO₄, 300 mM NaCl, wash buffer having the pH of 7.0, 250 mM imidazole, 50 mM NaH₂PO₄, 300 mM NaCl, and elution buffer having the pH of 7.0 were prepared. 20 mL of lysis buffer per 1 g of harvested cell pellet in an anaerobic chamber was mixed therewith and released with a pipette. Then, while checking the OD₆₀₀, the cells were lysed by ultrasonication until the value became 30% or less of the initial value, and were centrifuged at 11000 rpm and at a temperature of 4° C. for 20 minutes and only the supernatant was taken therefrom. 1 mL of Ni-NTA agarose based on 1 g of cell pellet, and supernatant were mixed and pipetted to bind the expressed protein for 15 minutes. The binding solution was poured onto the column, and 10 cv of wash buffer was poured and washed. For the following experiments, elution was performed with an elution buffer, and when operating a 10 L reactor, cells were lysed using a homogenizer.

### 3) Purification of RcFDH

50 mM NaH₂PO₄, 300 mM NaCl, lysis buffer having the pH of 8.0, 20 mM imidazole, 50 mM NaH₂PO₄, 300 mM NaCl, wash buffer having the pH of 8.0, 250 mM imidazole, 50 mM NaH₂PO₄, 300 mM NaCl, and elution buffer having the pH of 8.0 were prepared. 20 mL of lysis buffer per 1 g of harvested cell pellet in an anaerobic chamber was mixed therewith and released with a pipette. Cells were lysed by ultrasound for 5 minutes, and then subjected to centrifuging at 11000 rpm and at a temperature of 4° C. for 20 minutes and only supernatant was obtained therefrom. 1 mL of Ni-NTA agarose based on 1 g of cell pellet, and supernatant were mixed and pipetted to bind the expressed protein for 15 minutes. The binding solution was poured onto the column, and 10 cv of wash buffer was poured and washed. The resultant was eluted using elution butter and then used for the following experiments.

(6) Measurement of the concentrations of CODH and FDH

CODH concentration and TsFDH concentration were measured using Bradford assay. In the case of MeFDH I, an absorbance was 8.94 L/gm-cm (at 340 nm), and in the case of RcFDH, an absorbance was measured using Nanodrop at 340 nm and 169500 L/mol-cm.

### (7) Measurement of the Activity of CODH

50 mM HEPES, 2 mM DTE, and buffer having the having the pH of 8.0 were prepared in an anaerobic chamber. This buffer was placed in a serum bottle and purged with 100% CO gas for 1 hour. In an anaerobic chamber, 20 mM EV was added to the buffer prepared above, and 0.1 μg of CODH was injected thereto, and the activity was measured by measuring the change in absorbance at 578 nm while the temperature was maintained at a temperature of 30° C.

### (8) Measurement of FDH Activity

### 1) Measurement of MeFDH I Activity

20 μg of MeFDH I was added to 50 mM MOPS, 30 mM sodium formate, 0.5 mM NAD⁺, and 2 mL of buffer having the pH of 7.0, and the change in absorbance was measured while the temperature was maintained at a temperature of 30° C. at 340 nm, so as to measure the activity of the formic acid oxidation reaction.

### 2) Measurement of TsDFH Activity

20 μg of TsFDH was added to 100 mM sodium phosphate, 200 mM sodium formate, 2 mM NAD⁺, 2 mL of buffer having the pH of 6.5, and the change in absorbance was measured at 340 nm while the temperature was maintained at a temperature of 25° C. so as to measure the activity.

### 3) Measurement of RcFDH Activity

100 nM RcFDH was added to 100 mM Tris-HCl, 6 mM sodium formate, 2 mM NAD⁺, and a buffer having the pH of 9.0, and the change in absorbance was measured at 340 nm and at the temperature of 30° C. so as to measure the activity.

### Example 2. Composition of the CO Hydration Enzyme Reaction

Basically, the CO hydration enzyme reaction was performed in the presence of 200 mM Bis-Tris propane, 2 mM DTE, 2 UM resazurin, and a buffer having the pH of 6.5, and although the type and amount thereof varied depending on the experiment, and all experiments used CODH, FDH and an electron mediator. Unless gas was continuously introduced, the reaction was performed using 100% CO gas. In the experiments to select the types of CODH, FDH and electron mediator, 75 nM CODH, 150 nM FDH, and 5 mM electron mediator were used in a serum bottle. Activity measurement according to pH was tested by changing the pH by using 200 mM Bis-Tris propane buffer with a wide pH buffering range. In the case of MeFDH I, the CO₂ reduction reaction was measured. The EV which had been previously reduced with zinc and 50 mM sodium bicarbonate, was added to prepare CO₂. In an experiment using a 100 mL bubble column reactor, CODH 12000 U, FDH 400 U, and 5 mM EV were used, and in subsequent experiments, 1 mM EV was used. In the experiment in which LDG was used, after the experiment to obtain the appropriate EV concentration, EV was used at 0.1 mM. In the reusability-related experiment, 150 U CODH and 5 U FDH were put into a 10 mL serum bottle, and 500 KU CODH and 90 KU FDH were used in a 10 L reactor.

### Example 3. CO Hydration Reaction Results According to Types of CODH and FDH

The CO hydration reaction system configured in Example 2 was used, and the CO hydration reaction was carried out by changing the combination of CODH and FDH (based on the formic acid concentration measured 2 hours after the reaction, 5 mM ethyl viologen (EV) was used as the electron mediator).

TABLE 1

| Type | Type | Formic acid |
|---|---|---|
| of | of | production |
| FDH | CODH | concentration (mM) |
| MeFDH I | ChCODH | 40 |
| | II | |
| MeFDH I | ChCODH | 2 |
| | IV | |
| MeFDH I | ToCODH | 7 |
| RcFDH | ChCODH | 0 |
| | II | |
| RcFDH | ChCODH | 0 |
| | IV | |
| RcFDH | ToCODH | 0 |
| TsFDH | ChCODH | 0 |
| | II | |
| TsFDH | ChCODH | 0 |
| | IV | |
| TsFDH | ToCODH | 0 |

As a result, as can be seen in Table 1 above, only when MeFDH I was used as FDH, formic acid was produced, and in the case of other FDHs, formic acid was not produced at all. As a result, it was found that not all FDHs could not act due to the CO hydration reaction as a catalyst, and only certain FDHs could act.

However, as for CODH, in the case of all CODH, it was confirmed that formic acid has been produced. In the present embodiment, ChCODH II showed the best formic acid production ability. Therefore, the following experiments were carried out using MeFDH1 and ChCODH II as basic enzymes.

### Example 4: Comparison of Formic Acid Production According to the Type of Electron Mediator

Under the conditions used in Example 3, the enzymes ChCODH-II and MeFDH I were used together with electron mediators, and the results are shown in Table 2 below (measurement of formic acid concentration after 2 hours of reaction).

TABLE 2

| | Formic acid production |
|---|---|
| Type of electron mediator | concentration (mM) |
| Methyl viologen (MV) | 38 |
| Ethyl viologen (EV) | 40 |
| Benzyl viologen (BV) | 0 |
| Nicotinamide adenine dinucleotide (NAD) | 0 |
| Flavin adenine dinucleotide (FAD) | 0 |

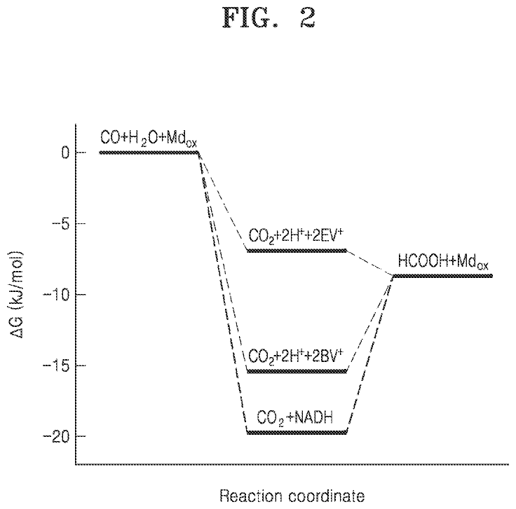

As a result, as can be seen in Table 2, it can be seen that the performance difference in mediating the CO hydration reaction is very large depending on the type of electron mediator. This difference could be explained by looking at the change in free energy shown in FIG. 2.

As can be seen in FIG. 2, in the case of BV and NAD, the thermodynamic state is very favorable for the CO oxidation reaction, but the reaction in which CO₂ is reduced to formic acid is thermodynamically disadvantageous because free energy is increased on the contrary, whereas in the case of EV, the free energy of CO oxidation and CO₂ reduction are continuously reduced, which can be interpreted that the production of formic acid is spontaneously favorable in the thermodynamic term.

### Example 5. Analysis of Optimal Activity Conditions for ChCODH-II and MeFDH-I

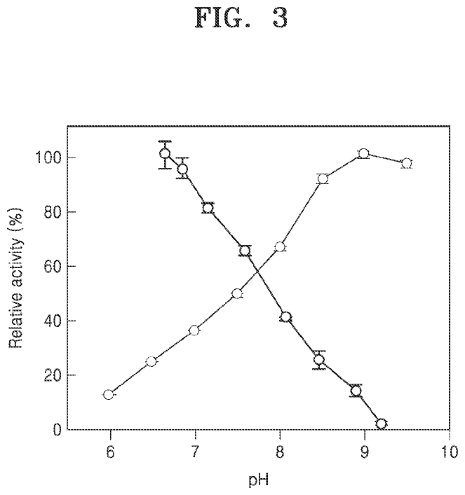

Since the CO hydration reaction needs to be performed under one condition, it was necessary to determine the initial input activity of enzymes in consideration of the effect of the two enzymes on the reaction conditions. Otherwise, it is difficult to produce formic acid smoothly due to an imbalance in the activity of enzymes. In order to solve this problem, as shown in FIG. 3, the activity according to the change in the hydrogen ion concentration (pH) was measured and indicated.

As a result, as shown in FIG. 3. it was confirmed that, under alkaline conditions, the activity of ChCODH II (black) was increased but the activity of MeFDH I (red) was rapidly decreased. In the case of CO₂, when dissolved in water, at the pH of 6.5 or higher, CO₂ does not exist and, instead, is present in the form of bicarbonate ions or carbonate ions. Accordingly, it was difficult to convert these ionized carbon dioxide molecules into formic acid by MeFDH1. Therefore, the reaction pH should be maintained below 6.5. At this time, however, there is a sharp decrease in the activity of ChCODH-II. Accordingly, when the CO hydration reaction is carried out under an environment of below 6.5, the initial input activity of ChCODH-II needs to be increased to expect the smooth production of formic acid.

### Example 6. Hourly Increase in the Concentration of Formic Acid Produced when CO-Containing Gas is Continuously Input into a 100 mL Bubble Column Reactor

A 100 mL bubble column reactor was operated while continuously introducing a mixed gas (using 50% CO, 50% CO₂ gas) and adjusting the pH (pH set to be 6.5, titrated with 2 N NaOH).

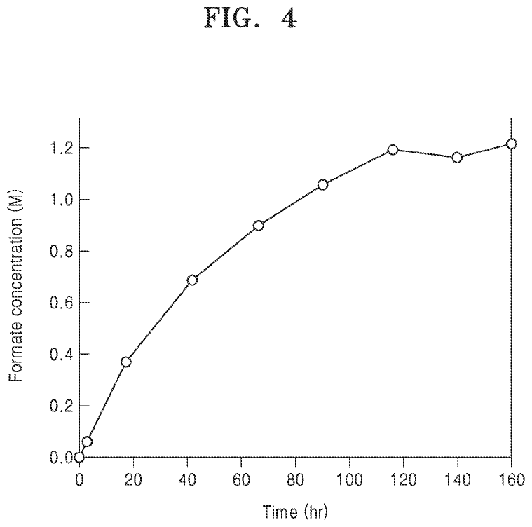

As a result, finally, more than 1 M formic acid was produced by the CO hydration enzyme reaction catalyzed by ChCODH II and MeFDH I (FIG. 4).

### Example 7. Test of Formic Acid Productivity Change in CO Hydration Reactor According to the Increase in the Flow Rate of Input Gas Containing CO

The productivity of formic acid was measured while increasing the flow rate of the input gas under the conditions of Example 6, is shown in Table 3 below (using the reactor volume of 100 mL). Carbon monoxide contained in the input gas was dissolved in water and then converted to formic acid by enzymes involved in the CO hydration reaction.

As can be seen in Table 3, when the flow rate of the gas is increased, the mass transfer rate of CO that is dissolved and transferred from the gas to the solution is increased, so it can be seen that the rate at which formic acid is generated by using the same is also increased.

TABLE 3

| | Formic acid production |
|---|---|
| Gas input flow (mL/min) | rate (mM/hr) |
| 200 | 10 |
| 500 | 43 |
| 1,000 | 78 |

### Example 8. Formic Acid Conversion Test Using Actual Waste Gas from the Steel Industry

The experiment was performed in the same manner as in Example 5, except that the CO hydration reaction was performed using the waste gas actually discharged from the steel industry instead of the conventional pure CO-containing gas. For this purpose, the gas composition components of the actual waste gas (LDG) used in the steel industry were analyzed and shown in Table 4. Referring to Table 4 below, it can be seen that the actual waste gas (LDG) included CO and CO₂ as the main components and a small amount of various compounds.

TABLE 4

| Materials | Amount |
|---|---|
| included | (%) |
| CO | 53.17 |
| CO₂ | 18.51 |
| H₂ | 1.43 |
| O₂ | 0.11 |
| N₂ | 26.77 |
| others | 0.01 |

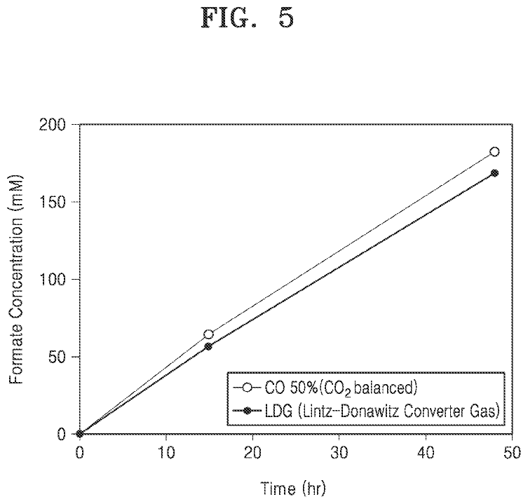

Using the same method as in Example 5, formic acid was produced using the actual steel industry waste gas having the composition of Table 4. Results thereof are shown in FIG. 5. Referring to FIG. 5, it can be see that in the case of actual waste gas, the production rate of formic acid was slightly small, but formic acid was generated almost similar to the case of pure crude gas. Through the experimental results of FIG. 5, it was confirmed that the activity of enzymes related to the CO hydration reaction was not significantly reduced by the unknown components contained in the waste gas, and formic acid was smoothly generated at a similar level.

### Example 9. Confirmation of Reusability of CO Hydration Reaction Enzyme Through Repeated Use of Used Enzyme

It was confirmed whether an enzyme could be used repeatedly under the same conditions as in Example 7.

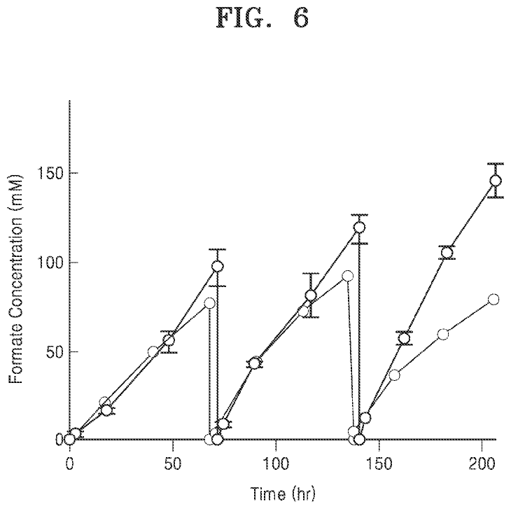

As a result, as shown in FIG. 6, it was confirmed that even when the enzyme was repeatedly applied to the gas generated in the steel industry, almost no decrease in the activity of the enzyme was observed and formic acid was generated. These results indicate that the enzymes proposed in the present disclosure are very stable and, in a state with high commercial applicability, can convert waste gas generated in the steel industry into formic acid without pre-treatment.

1. A composition for producing formic acid, the composition comprising carbon monoxide dehydrogenase and formate dehydrogenase.

2. The composition of claim 1, wherein the carbon monoxide dehydrogenase is derived from a microorganism belonging to at least one of Carboxydothermus sp. and Thermococcus sp.

3. The composition of claim 1, wherein the carbon monoxide dehydrogenase comprises at least one carbon monoxide dehydrogenase selected from a Carboxydothermus hydrogenoformans-derived carbon monoxide dehydrogenase II, a Carboxydothermus hydrogenoformans-derived carbon monoxide dehydrogenase IV, and a Thermococcus onnurineus-derived carbon monoxide dehydrogenase.

4. The composition of claim 1, wherein the formate dehydrogenase is derived from a microorganism belonging to at least one species selected from Methylobacterium sp., Thiobacillus sp. and Rhodobacter sp.

5. The composition of claim 1, wherein the formate dehydrogenase comprises at least one formate dehydrogenase selected from Methylobacterium extorquens-derived formate dehydrogenase I, Thiobacillus sp. KNK65MA-derived formate dehydrogenase, and Rhodobacter capsulatus-derived formate dehydrogenase.

6. The composition of claim 1, further comprising an electron mediator.

7. The method according to claim 6, wherein the electron mediator comprises at least one electron mediator selected from methyl viologen, ethyl viologen, benzyl viologen, nicotinamide adenine dinucleotide (NAD), and flavin adenine dinucleotide (FAD).

8. The composition of claim 1, wherein the carbon monoxide dehydrogenase and the formate dehydrogenase in the composition are present in a ratio of 1:1 to 1:3.

9. The composition of claim 6, wherein a pH of the composition is from 5.0 to 8.0.

10-12. (canceled)

13. A device for producing formic acid, the device comprising carbon monoxide dehydrogenase and formate dehydrogenase.

14-17. (canceled)

18. A method of producing a formic acid, the method comprising contacting a gas containing at least one molecule selected from carbon monoxide and carbon dioxide with carbon monoxide dehydrogenase and formate dehydrogenase.

19. The method of claim 18, wherein the contacting comprises: contacting the gas with carbon monoxide dehydrogenase; and
contacting, with the formate dehydrogenase, the gas which has been in contact with the carbon monoxide dehydrogenase.

20. The method of claim 18, wherein the gas is a continuously supplied gas.

21. The method of claim 18, wherein the gas is in contact with carbon monoxide dehydrogenase and formate dehydrogenase, simultaneously with the electron mediator.

22-24. (canceled)
